(19) United States
(12) Patent Application Publication
(10) Pub. No.: US 2024/0207391 A1
(43) Pub. Date: Jun. 27, 2024

(54) ENGINEERED INFLUENZA VIRUSES EXPRESSING SARS-COV-2 ANTIGENS, VACCINES AND METHODS OF MAKING AND USING THE SAME

(71) Applicant: Duke University, Durham, NC (US)

(72) Inventors: Nicholas Heaton, Durham, NC (US); Alfred Harding, Durham, NC (US); Brook Heaton, Durham, NC (US); Ryan Chaparian, Durham, NC (US)

(21) Appl. No.: 18/557,787

(22) PCT Filed: Apr. 27, 2022
(86) PCT No.: PCT/US2022/026574

§ 371 Date: Oct. 27, 2023

Related U.S. Application Data
(60) Provisional application No. 63/180,472, filed on Apr. 27, 2021.

(51) Int. Cl. A61K 39/215; A61K 39/00; A61K 39/145; A61P 37/04; C07K 14/005; C12N 7/00; C12N 9/24

(52) U.S. Cl. CPC A61K 39/215; A61K 39/145; A61P 37/04; C07K 14/005; C12N 7/00; C12N 9/2402; C12Y 302/01018; A61K 2039/5252; A61K 2039/5254; A61K 2039/5256; C07K 2319/02; C12N 2760/16122; C12N 2760/16134; C12N 2760/16151; C12N 2770/20022; C12N 2770/20034

(57) ABSTRACT

The present invention provides engineered polynucleotides encoding both the receptor binding domain (RBD) of a SARS-CoV-2 spike protein and an influenza hemagglutinin (HA) protein. Also provided are influenza viruses that comprise the engineered polynucleotides and express both the RBD and HA protein on their surface and methods for using these influenza viruses to generate an immune response to both influenza and SARS-CoV-2 in a subject.

### CROSS-REFERENCE TO RELATED APPLICATIONS

This patent application claims the benefit of priority of U.S. Provisional Patent Application No. 63/180,472, filed Apr. 27, 2021, which is incorporated herein by reference in its entirety.

### STATEMENT REGARDING FEDERALLY SPONSORED RESEARCH

This invention was made with government support under CEIRS contract number HHSN272201400005C awarded by the National Institute of Allergy and Infectious Diseases, National Institutes of Health, Department of Health and Human Services and under contract number 75N93019C00050 awarded by the National Institute of Allergy and Infectious diseases, National Institutes of Health. The government has certain rights in this invention.

### SEQUENCE LISTING

A Sequence Listing accompanies this application and is submitted as an ASCII text file of the sequence listing named “155554_00645_ST25.txt” which is 37,562 bytes in size and was created on Apr. 27, 2022. The sequence listing is electronically submitted via EFS-Web with the application and is incorporated herein by reference in its entirety.

### BACKGROUND

Severe acute respiratory syndrome coronavirus 2 (SARS-CoV-2) is a respiratory RNA virus that causes COVID-19, a disease that is similar in many respects to influenza virus-induced disease¹⁴. While several vaccines designed to vaccinate immunologically naïve people and provide protection against COVID-19 are currently in use, these vaccines are for the most part expensive, associated with significant side effects, and difficult to produce and distribute¹⁵⁻¹⁸. Further complicating vaccination efforts is the emergence of mutant strains of SARS-CoV-2, such as the Delta and Omicron variants, that have been associated with reduced vaccine efficacy¹⁹⁻²¹. Additionally, protective immunity against human coronaviruses in general, is thought to be relatively short lived²²⁻²⁵. Thus, there is a remaining need in the art for a cost-effective, scalable, and safe vaccine to periodically boost immunity against SARS-CoV-2.

### SUMMARY

In a first aspect, the present invention provides engineered polynucleotides comprising: (a) a first polynucleotide encoding from 5′ to 3′ relative to a sense strand a portion of an influenza neuraminidase (NA) protein comprising a cytoplasmic tail and a transmembrane domain fused to a receptor binding domain (RBD) of a SARS-CoV-2 spike protein; and (b) a second polynucleotide encoding an influenza hemagglutinin (HA) protein. Within the engineered polynucleotides, the first polynucleotide is linked to the second polynucleotide by a third polynucleotide encoding a linker peptide.

In a second aspect, the present invention provides plasmids comprising the engineered polynucleotides described herein. As used herein, a “plasmid” is a circular double-stranded DNA molecule that can replicate independently of the genome in a cell. The plasmid may further comprise at least one promoter operably linked to the polynucleotides to allow for expression of the proteins. The plasmid may also comprise a promoter operably linked to the polynucleotides to allow for production of the negative sense RNA encoding a viral segment. Plasmids may comprise both of these promoters to allow for production of both sense and antisense strands of RNA encoding the proteins on the sense strand and encoding the viral segment for inclusion in recombinant influenza virus in the antisense strand.

In a third aspect, the present invention provides plasmid compositions comprising a plasmid described herein and plasmids encoding influenza virus segments 1, 2, 3, 5, 6, 7, and 8.

In a fourth aspect, the present invention provides cells comprising a plasmid composition described herein, as well as influenza viruses produced from these cells.

In a fifth aspect, the present invention provides influenza viruses comprising the engineered polynucleotide described herein.

In a sixth aspect, the present invention provides pharmaceutical compositions comprising an influenza virus described herein and a pharmaceutically acceptable carrier.

In a seventh aspect, the present invention provides methods for generating an immune response to both influenza and SARS-CoV-2 in a subject. The methods comprise administering a therapeutically effective amount of an influenza virus or pharmaceutical composition described herein to the subject.

In an eighth aspect, the present invention provides methods for producing an influenza virus. The methods comprise (a) transfecting a plasmid composition described herein into a cell; (b) incubating the transfected cell; and (c) harvesting the influenza virus produced by the cells. These methods produce influenza viruses that express both the RBD of the SARS-CoV-2 spike protein and the influenza hemagglutinin protein on their surface.

### BRIEF DESCRIPTION OF THE DRAWINGS

FIG. 1 shows the generation of an influenza A virus (IAV) that encodes a vaccine antigen in the HA segment. (A) Diagram showing genetic modulation of the HA segment to enable insertion of a foreign ORF. The SARS-CoV-2 RBD was fused to the NA transmembrane domain and a PTV1-2A site was introduced to allow for co-translation of the RBD and HA, forming the “TM-RBD-HA” virus. (B) RT-PCR analysis of segment 4 of the WT and TM-RBD-HA viruses. (C) Immunofluorescence microscopy images of unpermeablized MDCK cells infected without virus (top), with WT virus (middle), or with TM-RBD-HA virus (bottom). Cells were subsequently stained with antibodies/dyes against nuclei (first column), HA (second column), or SARS-CoV-2 RBD (third column). Merged images are presented in the fourth column. Scale bar indicates 100 μm. (D) HA assays of WT and TM-RBD-HA viruses after growth in embryonated chicken eggs for 72 hours. Each dot represents an individual egg, n=4. (E) Plaque assays of WT and TM-RBD-HA viruses grown in embryonated chicken eggs at 24, 48, and 72 hours, n=4. (F) Plaque assays of WT and TM-RBD-HA viruses after growth in embryonated chicken eggs for 72 hours from (E). Each dot represents an individual egg, n=4. (G) RT-PCR analysis of segment 4 from WT and TM-RBD-HA viruses after 10 passages on MDCK cells. PO indicates the stock of virus used for the experiment. Statistical analyses were performed using unpaired Mann-Whitney tests. For all panels, asterisks indicate P-values<0.05. Error bars indicate SEM.

FIG. 2 demonstrates that the SARS-CoV-2 RBD is stably incorporated into IAV particles without disrupting other viral envelope proteins. (A) Western blot analysis of WT and TM-RBD-HA viruses. Samples were normalized via M1 protein signal using pixel densitometry. (B) ELISAs (left) against whole virus particles using the PY102 anti-HA antibody and area under the curve analysis (right, n=4). (C) ELISAs (left) against whole virus particles using a SARS-CoV-2 RBD antibody (binds a non-structural epitope) and area under the curve analysis (right, n=4). (D) ELISAs (left) against whole virus particles using a SARS-CoV-2 neutralizing antibody (DH1041, binds a structural epitope on the RBD) and area under the curve analysis (right, n=4). (E) Same analysis as in (D) using a different conformation-specific (“con. specific”) SARS-CoV-2 neutralizing antibody, DH1044 (n=4). Statistical analyses were performed using unpaired Mann-Whitney tests. For all panels, asterisks indicate P-values<0.05. Error bars indicate SEM.

FIG. 3 demonstrates that the TM-RBD-HA virus is attenuated in vitro and in vivo and does not functionally bind ACE2. (A) Growth curves (n=3) and endpoint titers (n=4) of WT and TM-RBD-HA viruses on A549 and A549-ACE2 cells. (B) Transduction of A549 and A549-ACE2 cells with pseudotyped lentiviral particles; relative light units (RLU) were measured as a proxy for cell entry (n=4). (C) Infection of SialidaseA-treated A549-ACE2 cells with WT and TM-RBD-HA viruses. (D) Number of infected cells from 5 representative sections from (C). (E) plaque reduction neutralization tests (PRNTs) using anti-HA PY102 with WT and TM-RBD-HA viruses (n=4). (F) Bodyweights of K18-hACE2 mice infected with WT or TM-RBD-HA viruses (n=4). (G) Survival of mice from (F). Statistical analyses were performed using unpaired Mann-Whitney tests for panels A, B, and D, and Mantel-Cox tests for panel G. For survival plots, statistical tests were applied to compare the survival rates of mice infected with the same amount of WT or TM-RBD-HA virus. For all panels, asterisks indicate the following P-values: **<0.01, *<0.05, ns=not significant. Dotted lines represent PRNT₅₀ or humane endpoints. Error bars indicate SEM.

FIG. 4 demonstrates that live-attenuated vaccination of mice with TM-RBD-HA virus elicits neutralizing antibody responses against both IAV and SARS-CoV-2. (A) Diagram illustrating vaccination and sample collection timepoints. (B) ELISAs against purified soluble HA protein using sera from vaccinated C57BL/6J mice comparing responses between inactivated virus vaccine (n=5) and live, sub-lethal virus vaccine (LAIV, n=4). (C) Area under the curve analysis of (B). (D) ELISAs against purified soluble RBD protein using sera from K18-hACE2 vaccinated mice comparing responses between inactivated virus vaccine (n=4) and LAIV (n=8 except TM-RBD-HA, n=7). (E) Area under the curve analysis for (D). (F) PRNTs with sera from vaccinated C57BL/6J mice against live A/Puerto Rico/8/1934 virus (n=4). (G) Neutralization titer quantification of PRNTs from (F). (H) PRNTs with sera from vaccinated K18-hACE2 mice against live SARS-CoV-2 USA-WA/2020 virus (n=8). (I) Neutralization titer quantification of PRNTs from (H). Statistical analyses were performed using unpaired Mann-Whitney tests. For all panels, asterisks denote the following P-values: ***<0.001, **<0.01, *<0.05, ns=not significant. Error bars indicate SEM. Error bars corresponding to values less than 0 have been clipped from panel F. Dotted lines represent limit of detection (LOD) or PRNT₅₀; for undetectable samples, data points have been assigned the LOD value.

FIG. 5 demonstrates that live-attenuated vaccination with TM-RBD-HA virus provides protection against IAV and SARS-CoV-2 challenge in mice. (A) Experimental design displaying vaccination/challenge timepoints. (B) Bodyweights of C57BL/6J mice vaccinated with either WT virus, TM-RBD-HA virus, or BSA and challenged with a lethal dose of A/Puerto Rico/8/1934 (n=5). (C) Survival of mice from (B). (D) Quantification of IAV PFUs in lung homogenates from vaccinated/challenged mice (n=5). (E) Lung tissue histology (H&E staining) of mice challenged with A/Puerto Rico/8/1934 (representative images, n=3). (F) Bodyweights of K18-hACE2 mice vaccinated with either WT virus, TM-RBD-HA virus, or BSA and challenged with a lethal dose of SARS-CoV-2 USA-WA/2020, (n=8, except TM-RBD-HA, n=7). (G) Survival of mice from (F). (H) Quantification of CoV PFUs in lung homogenates from vaccinated/challenged mice (n=4). (I) Lung tissue histology (H&E staining) of mice challenged with SARS-CoV-2 USA-WA/2020 (representative images, n=4). For all microscopy images, scale bars indicate 200 μm and 50 μm for 10× and 40× magnified samples, respectively. Unpaired Mann-Whitney tests and Mantel-Cox tests were used for panels D/H and C/G, respectively. For survival plots, statistical tests were applied to compare WT/TM-RBD-HA groups against the BSA group. For all panels, asterisks denote the following P-values: ***<0.001, **<0.01, *<0.05. Error bars indicate SEM. Dotted lines represent limit of detection (LOD) or humane endpoints; for undetectable samples, data points have been assigned the LOD value.

FIG. 6 shows a sandwich ELISA analysis of WT/TM-RBD-HA viruses in which the DH1041 antibody was used to capture the virus and the PY102 antibody was subsequently used to detect the virus, n=4. Statistical analyses were performed using unpaired Mann-Whitney tests. Asterisks indicate P-values<0.05. Error bars indicate SEM.

FIG. 7 shows the experimental design and vaccination schedule for the inactivated virus. At the start of the experiment, C57BL/6J or K18-hACE2 mice were vaccinated with either BSA or inactivated WT/TM-RBD-HA virus. At day 21, serum was collected for analysis and mice were boosted with an additional dose of either BSA or inactivated WT/TM-RBD-HA virus. At day 35, serum was collected, and mice were challenged with a lethal dose of IAV or SARS-CoV-2. During days 35-49, the mice were monitored for mortality after lethal challenge. For certain analyses, lungs were harvested 5 days after lethal challenge.

FIG. 8 demonstrates that vaccination of mice with inactivated TM-RBD-HA virus elicits neutralizing antibody responses and protective immunity against IAV. (A) ELISAs (left) against purified soluble HA and area under the curve analysis (right) using sera from C57BL/6J mice vaccinated with inactivated virus (n=5). (B) ELISAs (left) against purified soluble HA and area under the curve analysis (right) using sera from K18-hACE2 mice vaccinated with inactivated virus (n=4). (C) PRNTs with sera from vaccinated C57BL/6J mice against live A/Puerto Rico/8/1934 virus (n=5). (D) Bodyweights of C57BL/6J mice vaccinated with WT virus, TM-RBD-HA virus, or BSA and challenged with a lethal dose (500 PFU) of A/Puerto Rico/8/1934 (n=5). (E) Survival of mice from (D). (F) Quantification of IAV PFUs in lung homogenates from vaccinated/challenged C57BL/6J mice (n=4). (G) Lung tissue histology (H&E staining) of vaccinated/challenged C57BL/6J mice (representative images, n=3). Statistical analyses were performed using unpaired Mann-Whitney tests for panels A, B, and F and Mantel-Cox tests for panel E. For survival plots, statistical tests were applied to compare WT/TM-RBD-HA groups against the BSA group. For all panels, asterisks denote the following P-values: **<0.01, *<0.05, ns=not significant. For all microscopy images, the scale bar indicates 200 μm. Error bars indicate SEM. Dotted lines represent limit of detection (LOD), PRNT₅₀, or humane endpoints; for undetectable samples, data points have been assigned the LOD value.

FIG. 9 demonstrates that vaccination of mice with inactivated TM-RBD-HA virus fails to elicit neutralizing antibody responses and is insufficient to protect against SARS-CoV-2. (A) ELISAs (left) against purified soluble RBD protein and area under the curve analysis (right) using sera from C57BL/6J mice vaccinated with inactivated virus (n=5). (B) ELISAs (left) against purified soluble RBD protein and area under the curve analysis (right) using sera from K18-hACE2 mice vaccinated with inactivated virus (n=4). (C) Sera from mice vaccinated with inactivated TM-RBD-HA virus (n=4) were used for PRNTs against SARS-CoV-2 USA-WA/2020. (D) Bodyweights of K18-hACE2 mice vaccinated with WT, TM-RBD-HA, or BSA and challenged with a lethal dose (3×10⁴ PFU) of SARS-CoV-2 USA-WA/2020 (n=8, except TM-RBD-HA, n=7). (E) Survival of mice from (D). (F) Quantification of SARS-CoV-2 PFUs in lung homogenates from vaccinated/challenged K18-hACE2 mice (n=4). (G) Lung tissue histology (H&E staining) of vaccinated/challenged K18-hACE2 mice (representative images, n=3). Statistical analyses were performed using unpaired Mann-Whitney tests for panels A, B, and F and Mantel-Cox tests for panel E. For survival plots, statistical tests were applied to compare WT/TM-RBD-HA groups to the BSA group. For all panels, asterisks denote the following P-values: **<0.01, ns=not significant. For all microscopy images, the scale bar indicates 200 μm. Error bars indicate SEM. Dotted lines represent limit of detection (LOD), PRNT₅₀, or humane endpoints; for undetectable samples, data points have been assigned the LOD value.

FIG. 10 demonstrates that a prime (LAIV) boost (inactivated) vaccination scheme improves HA/RBD immune responses in both C57BL/6J and K18-hACE2 mice. (A) ELISAs (left) against purified soluble HA protein and area under the curve analysis (right) using sera from C57BL/6J mice vaccinated with live virus (n=4). (B) ELISAs (left) against purified soluble HA protein and area under the curve analysis (right) using sera from K18-hACE2 mice vaccinated with live virus (n=8, n=7 for TM-RBD-HA). (C) ELISAs (left) against purified soluble RBD protein and area under the curve analysis (right) using sera from C57BL/6J mice vaccinated with live virus (n=4). (D) ELISAs (left) against purified soluble RBD protein and area under the curve analysis (right) using sera from K18-hACE2 mice vaccinated with live virus (n=8). Statistical analyses were performed using unpaired Mann-Whitney tests. For all panels, asterisks denote the following P-values: ***<0.001,*<0.05, ns=not significant. Error bars indicate SEM.

### DETAILED DESCRIPTION

The present invention provides engineered polynucleotides encoding both the receptor binding domain (RBD) of a SARS-CoV-2 spike protein and an influenza hemagglutinin (HA) protein. Also provided are influenza viruses that comprise the engineered polynucleotides and express both the RBD and HA protein on their surface and methods for using these influenza viruses to generate an immune response to both influenza and SARS-CoV-2 in a subject.

Every year, influenza virus vaccines are produced, distributed, and administered in sufficient numbers to vaccinate the global population¹. Influenza vaccines are widely accepted to be safe and efficacious, but they must be reformatted annually due to viral antigenic drift²⁻⁴. Several types of influenza vaccines are currently in clinical use, including: purified subunit vaccines, inactivated vaccines, and live-attenuated influenza vaccines (LAIV)¹. The FDA-approved subunit vaccines consist of a recombinantly expressed hemagglutinin (HA) protein and, like all current vaccination strategies, they are primarily designed to elicit virus-neutralizing antibodies⁵. Inactivated influenza vaccines consist of chemically inactivated viruses, are replication-incompetent, and represent the most common formulation for vaccination against influenza. LAIVs are replication competent but are built on cold-adapted backbones that possess mutations that limit viral replication above 33° C. and prevent infection of the lower respiratory tract. LAIVs are thought to mediate superior stimulation of CD4+/CD8+ T cells as compared to traditional inactivated influenza vaccines and they uniquely elicit IgA antibodies⁶⁻⁹. Thus, several effective approaches have been developed to induce influenza-directed immunity.

Because of their broad use and immunogenicity, influenza viruses have great potential for use as a vaccine platform. Reverse genetic approaches¹⁰ have allowed non-influenza proteins and immune epitopes to be introduced into influenza viral strains¹¹⁻¹³. The resulting recombinant influenza strains could serve as vehicles for introducing these heterologous antigens to the immune system. Thus, leveraging the existing influenza virus vaccine production infrastructure to produce recombinant viral strains that express antigens from other pathogens may be a practical, cost-effective approach for generating easily implemented combination vaccines or boosters.

Thus, to test the ability of a platform-based solution to boost immunity against SARS-CoV-2 and associated variants, the present inventors have developed an influenza virus-based vaccine that comprises antigens from both influenza A virus (IAV) and SARS-CoV-2. In the Examples, they demonstrate that this vaccine stably expresses and packages a small but immunogenic domain of the SARS-CoV-2 spike protein and that it elicits neutralizing antibodies and provides protection against lethal challenge with both viruses in mouse models of infection. Thus, the inventors have generated a combination vaccine that can be manufactured in the same way that most influenza virus vaccines are manufactured. To limit the cost and human vaccine burden, a seasonal IAV/SARS-CoV-2 combination vaccine could replace the standard seasonal IAV vaccine and provide protection against novel variants of both IAV and SARS-CoV-2. Advantageously, this approach would not require additional vaccine manufacturing or distribution facilities.

### Engineered Polynucleotides:

In a first aspect, the present invention provides engineered polynucleotides comprising: (a) a first polynucleotide encoding from 5′ to 3′ relative to a sense strand of the polynucleotide a portion of an influenza neuraminidase (NA) protein comprising a cytoplasmic tail and a transmembrane domain fused to a receptor binding domain (RBD) of a SARS-CoV-2 spike protein; and (b) a second polynucleotide encoding an influenza hemagglutinin (HA) protein. Within the engineered polynucleotides, the first polynucleotide is linked to the second polynucleotide by a third polynucleotide encoding a linker peptide.

The terms “polynucleotide,” “oligonucleotide,” and “nucleic acid” are used interchangeably to refer a polymer of DNA or RNA. A polynucleotide may be single-stranded or double-stranded and may represent the sense or the antisense strand. A “sense strand” or “positive-sense strand” is a strand of polynucleotide that has the same sequence as the transcribed mRNA, whereas the “antisense strand” or “negative-sense strand” is the reverse complementary strand that is used as a template during transcription or in the case of a negative sense virus like IAV may represent the viral genomic segment. A polynucleotide may be synthesized or obtained from a natural source. A polynucleotide may contain natural, non-natural, or altered nucleotides, as well as natural, non-natural, or altered internucleotide linkages. The term polynucleotide encompasses constructs, plasmids, vectors, viruses and the like.

The genomes of all influenza viruses are composed of eight single-stranded RNA segments that are “negative-sense”, meaning that they must be copied into positive-sense molecules to direct the production of proteins. Thus, in some embodiments, the engineered polynucleotides comprise single-stranded, negative-sense RNA. In other embodiments, the engineered polynucleotides comprise DNA. For example, the engineered polynucleotide may be part of a plasmid that is used to produce a virus in a cell via viral rescue, as discussed below.

In some embodiments, the engineered polynucleotide is part of a viral segment. The genomes of RNA viruses are commonly divided into multiple distinct RNA molecules, which are referred to as “viral segments”. For example, the genomes of influenza A viruses contain eight segments of single-stranded RNA that each encode 1-2 proteins. Specifically, segment 1 encodes polymerase basic protein 2 (PB2), segment 2 encodes polymerase basic protein 1 (PB1), segment 3 encodes polymerase acidic protein (PA), segment 4 encodes hemagglutinin (HA), segment 5 encodes nucleoprotein (NP), segment 6 encodes neuraminidase (NA), segment 7 encodes matrix protein 1 (M1) and matrix protein 2 (M2), and segment 8 encodes non-structural protein 1 (NS1) and non-structural protein 2 (NS2; also referred to as NEP). In the Examples, the inventors generated an engineered polynucleotide from segment 4 of an influenza A virus (IAV) genome. Thus, in some embodiments, the engineered polynucleotide is part of segment 4 from an influenza virus. In other embodiments, the polynucleotide is part of segment 6 from an influenza virus. Suitable methods of engineering IAV segment 6 are described in PCT Application No. PCT/US2017/041737, which is hereby incorporated by reference in its entirety.

In some embodiments, the engineered polynucleotides further comprise an influenza virus packaging signal. As used herein, an “influenza virus packaging signal” is a cis-acting sequence or set of sequences that is included in a viral genome segment and is required to ensure that the segment is packaged into a viral particle during viral production. Influenza virus packaging signal(s) have been identified for each influenza A virus segment (J Virol 86:7043-7051, 2012). In some embodiments, the viral packaging signal comprises SEQ ID NO: 3, i.e., the viral packing signal that was used in the Examples.

In the Examples, the inventors tested an engineered polynucleotide that comprised from 5′ to 3′ on the sense strand: (a) a viral 5′ untranslated region (UTR); (b) a viral packaging signal; (c) the first polynucleotide; (d) the third polynucleotide; (e) the second polynucleotide; and (f) a viral 3′ UTR. Thus, in some embodiments, the engineered polynucleotide comprises these components in this particular 5′ to 3′ order for the sense strand.

The genome segments of influenza viruses comprise a central open reading frame (in the negative-sense orientation) flanked at both ends by viral UTRs, i.e., a 5′ UTR and a 3′ UTR. The viral UTRs play important roles in the genome packaging of influenza viruses.

The polynucleotides of the present invention encode polypeptides. The terms “polypeptide,” “protein,” and “peptide” are used interchangeably herein to refer to a series of amino acid residues connected by peptide bonds between the alpha-amino and carboxy groups of adjacent residues, forming a polymer of amino acids. Polypeptides may include modified amino acids and amino acid analogs.

The engineered polynucleotides of the present invention encode the RBD of a SARS-CoV-2 spike protein. Severe acute respiratory syndrome coronavirus 2 (SARS-CoV-2) is a strain of coronavirus that causes the respiratory illness COVID-19 (coronavirus disease 2019). SARS-CoV-2 contains four structural proteins, i.e., the spike (S), envelope (E), membrane (M), and nucleocapsid (N) proteins. The spike protein mediates viral entry into host cells by binding the host receptor angiotensin-converting enzyme 2 (ACE2) via the receptor-binding domain (RBD) in its S1 subunit and then fusing the virus and host membrane via its S2 subunit. Thus, the spike protein has served as the primary target for the development of vaccines against SARS-CoV-2.

The inventors opted to use the RBD of the spike protein as a vaccine antigen. The RBD that they utilized comprises residues Arg319-Phe541 of the full-length spike protein expressed by the Wuhan variant of SARS-CoV-2 (RBD: SEQ ID NO: 11; full-length spike protein: SEQ ID NO: 15). Thus, in some embodiments, the RBD comprises SEQ ID NO: 11 or a sequence that has at least about 60%, 65%, 70%, 75%, 80%, 85%, 90%, 95%, 98%, or 99% sequence identity to SEQ ID NO: 11. However, the RBD of the spike protein from a different variant of SARS-CoV-2 may also be used with the present invention. Other exemplary RBDs include the RBDs from the SARS-CoV-2 Beta variant (SEQ ID NO: 20), the SARS-CoV-2 Gamma variant (SEQ ID NO: 21), the SARS-CoV-2 Delta variant (SEQ ID NO: 22), and the SARS-CoV-2 Omicron variant (SEQ ID NO: 23). The antigen used in the polynucleotides, compositions and viruses provided herein may have at least about 60%, 65%, 70%, 75%, 80%, 85%, 90%, 95%, 98%, or 99% sequence identity to these RBD variants. The RBDs of many spike proteins have been annotated and one of skill in the art would be able to identify the RBD within a full-length spike protein based on a structural/functional analysis or based on sequence homology with SEQ ID NO: 11. Suitable RBDs may comprise about 150 to about 250, preferably about 200 to about 250 amino acid residues of the full-length spike protein. As noted above, the spike protein of SARS-CoV-2 has already mutated substantially over the first two years of the pandemic, and those of skill in the art will recognize that it is likely this protein will continue to evolve and acquire additional mutations over time. Thus, the inventors and those of skill in the art expect that both the HA sequence and the RBD sequence used in the polynucleotides, plasmids, and viruses described herein will need to be substituted on a regular basis to effectively vaccinate against the circulating strains of influenza and SARS-CoV-2.

“Percentage of sequence identity” is determined by comparing two optimally aligned sequences over a comparison window. The aligned sequences may comprise additions or deletions (i.e., gaps) relative to each other for optimal alignment. The percentage is calculated by determining the number of matched positions at which an identical nucleic acid base or amino acid residue occurs in both sequences, dividing the number of matched positions by the total number of positions in the window of comparison and multiplying the result by 100 to yield the percentage of sequence identity. Protein and nucleic acid sequence identities are evaluated using the Basic Local Alignment Search Tool (“BLAST”), which is well known in the art (Proc. Natl. Acad. Sci. USA (1990) 87: 2267-2268; Nucl. Acids Res. (1997) 25: 3389-3402). The BLAST programs identify homologous sequences by identifying similar segments, which are referred to herein as “high-scoring segment pairs”, between a query amino acid or nucleic acid sequence and a test sequence which is preferably obtained from a protein or nucleic acid sequence database. Preferably, the statistical significance of a high-scoring segment pair is evaluated using the statistical significance formula Proc. Natl. Acad. Sci. USA (1990) 87: 2267-2268), the disclosure of which is incorporated by reference in its entirety. The BLAST programs can be used with the default parameters or with modified parameters provided by the user.

To ensure that the RBD is tethered to the viral surface, the inventors expressed this peptide as a fusion protein with a portion of an influenza NA protein that comprises the transmembrane domain and the cytoplasmic tail of NA. The NA transmembrane domain anchors the RBD-NA fusion protein in the cell membrane and the NA cytoplasmic tail is believed to allow the fusion protein to be packaged into influenza virus particles.

Neuraminidase (NA) is an enzymatic protein found on the surface of influenza viruses. The NA peptide used with the present invention may be a portion of an NA protein from any of the 11 known NA subtypes including, without limitation, N1, N2, N3, N4, N5, N6, N7, N8, N9, N10, or N11. Suitably, the NA protein is from the subtype N1, N2, N3, or N7. The transmembrane domain and cytoplasmic tail are found on the N-terminus of the full-length NA protein. Thus, in some embodiments, the portion of the influenza NA protein comprises amino acids 1-40 of an influenza NA protein. In some embodiments, the portion of the influenza NA protein comprises SEQ ID NO: 10, i.e., the NA peptide sequence that was used in the Examples. In some embodiments, the NA peptide comprises a sequence that has at least about 60%, 65%, 70%, 75%, 80%, 85%, 90%, 95%, 98%, or 99% sequence identity to SEQ ID NO: 10. However, an NA protein from a different influenza virus may also be used with the present invention. The transmembrane domain and cytoplasmic tail of many NA proteins have been annotated and one of skill in the art would be able to determine the portion of an NA protein that comprises these domains based on a structural/functional analysis or based on sequence homology with SEQ ID NO: 10.

The engineered polynucleotides of the present invention encode a linker peptide that separates the encoded RBD-NA fusion protein (also referred to herein as “TM-RBD”) from the encoded HA protein. As used herein, the term “linker peptide” refers to a peptide sequence that bridges two protein components in a fusion protein. The linker peptide comprises 1 or more amino acid residues, preferably 1, 2, 3, 4, 5, 6, 7, 8, 9, 10, 11, 12, 13, 14, 15, 16, 17, 18, 19, or 20 or more residues.

In the Examples, the inventors included a protein tag in the linker peptide for easy detection of the protein products produced from the engineered polynucleotide. Thus, in some embodiments, the linker peptide comprises a protein tag. As used herein, a “protein tag” is a peptide sequence that is included in a fusion protein to facilitate detection or isolation of the fusion protein. Suitable protein tags include, but are not limited to, 6-Histidine (His), hemagglutinin (HA), cMyc, GST, flag, V5, and NE tags. In some embodiments, the protein tag is a flag tag. In some embodiments, the flag tag comprises SEQ ID NO: 12, i.e., the flag tag sequence that was used in the Examples.

In the Examples, the inventors also included a self-cleaving 2A peptide in the linker peptide to ensure that the RBD and HA proteins were co-translationally separated. Thus, in some embodiments, the linker peptide comprises a “self-cleaving polypeptide”, i.e., a polypeptide that results in the separation of the protein components flanking it. Suitable self-cleaving polypeptides include 2A peptides, which induce ribosomal skipping during translation of a protein. 2A polypeptides are known in the art and are described, for example, in PLOS ONE, 6(4), e18556, 2011. Exemplary 2A peptides include, without limitation, FMDV 2A, equine rhinitis A virus (ERAV) 2A (E2A), porcine teschovirus-1 2A (PTV1-2A), and Thoseaasigna virus 2A (T2A). Preferably, 2A peptide includes a PTV1-2A motif. In some embodiments, the 2A peptide comprises SEQ ID NO: 13, i.e., the 2A peptide sequence that was used in the Examples. In some embodiments, the 2A peptide comprises a sequence having at least 90%, 95%, 98%, or 99% sequence identity to SEQ ID NO: 13.

The engineered polynucleotides of the present invention encode an influenza HA protein. Hemagglutinin (HA) is a glycoprotein found on the surface of influenza viruses. The HA protein used with the present invention may be from any influenza virus, preferably from an influenza A or influenza B virus. The HA protein may be from any IAV HA subtype, including the H1, H2, H3, H4, H5, H6, H7, H8, H9, H10, H11, H12, H13, H14, H15, H16, H17, or H18 subtype. Suitably, the HA protein may be from HA subtype 1 (H1) or HA subtype 3 (H3), i.e., the IAV HA subtypes that are used in the human seasonal vaccine. In some embodiments, the influenza HA protein comprises SEQ ID NO: 14, i.e., the HA subtype 1 protein sequence that was used in the Examples.

In the Examples, the inventors tested an engineered polynucleotide that comprises SEQ ID NO: 1, which is derived from segment 4 of the IAV genome. Thus, in some embodiments, the engineered polynucleotide comprises SEQ ID NO: 1 or a sequence having at least 60% sequence identity to SEQ ID NO: 1. For example, the engineered polynucleotide may comprise a sequence having at least 60%, 65%, 70%, 75%, 80%, 85%, 90%, 95%, 98%, or 99% sequence identity to SEQ ID NO: 1. From 5′ to 3′, SEQ ID NO: 1 comprises: the viral 5′ UTR of SEQ ID NO: 2, the viral packaging signal of SEQ ID NO: 3, a Kozak signal (i.e., GCCACC), SEQ ID NO: 4 which encodes a portion of a NA polypeptide (amino acid sequence: SEQ ID NO: 10), SEQ ID NO: 5 which encodes the RBD of a spike protein (amino acid sequence: SEQ ID NO: 11), SEQ ID NO: 6 which encodes a flag tag (amino acid sequence: SEQ ID NO: 12), SEQ ID NO: 7 which encodes a 2A self-cleaving peptide (amino acid sequence: SEQ ID NO: 13), and SEQ ID NO: 8 which encodes an HA protein (amino acid sequence: SEQ ID NO: 14).

The engineered polynucleotides may be optimized for the codon usage of a specific influenza virus. The genomes of influenza viruses have low GC content and preferentially utilize different codons than those used in eukaryotic genomes. Thus, to enhance the expression and stability of polynucleotides, the polynucleotide sequences may be optimized using an optimization tool such as the publicly available Codon Optimization On-Line (COOL) tool or the OPTIMIZER tool. For example, codon usage of the influenza A virus may be determined using the following table (Table 1) from the Codon Usage Database.

TABLE 1
Codon usage of the influenza A virus
Coding GC 43.85% 1st letter GC 46.79% 2nd letter GC 40.45% 3rd letter GC 44.30%

| | | | | | | | |
|---|---|---|---|---|---|---|---|
| UUU | 17.8(293396) | UCU | 12.8(210832) | UAU | 14.2(233240) | UGU | 7.6(125361) |
| UUC | 20.2(332322) | UCC | 11.0(181613) | UAC | 12.6(207199) | UGC | 11.2(184026) |
| UUA | 8.1(133967) | UCA | 19.0(312168) | UAA | 1.2(19221) | UGA | 0.8(12569) |
| UUG | 14.6(240030) | UCG | 3.8(61962) | UAG | 0.5(8905) | UGG | 16.4(270035) |
| CUU | 16.8(276221) | CCU | 11.3(185417) | CAU | 9.9(162471) | CGU | 2.0(33533) |
| CUC | 12.2(200260) | CCC | 7.3(119819) | CAC | 7.2(118729) | CGC | 3.2(52445) |
| CUA | 13.3(219434) | CCA | 14.7(242107) | CAA | 22.5(371086) | CGA | 6.1(99775) |
| CUG | 14.8(243631) | CCG | 4.7(76633) | CAG | 18.4(302804) | CGG | 5.6(91717) |
| AUU | 23.7(389594) | ACU | 18.3(301672) | AAU | 31.3(515196) | AGU | 14.4(237994) |
| AUC | 18.1(298586) | ACC | 13.2(217249) | AAC | 23.5(387110) | AGC | 15.3(252010) |
| AUA | 24.4(402080) | ACA | 27.7(455971) | AAA | 35.9(591384) | AGA | 31.7(521693) |
| AUG | 37.8(622324) | ACG | 4.9(80019) | AAG | 22.2(365188) | AGG | 18.2(299901) |
| GUU | 13.4(220590) | GCU | 15.4(254219) | GAU | 26.1(429380) | GGU | 9.9(162838) |
| GUC | 11.1(183609) | GCC | 12.6(207403) | GAC | 20.2(332513) | GGC | 9.0(148927) |
| GUA | 12.5(205254) | GCA | 24.2(398856) | GAA | 40.5(667159) | GGA | 29.8(491304) |
| GUG | 19.6(323574) | GCG | 4.5(74308) | GAG | 31.2(513739) | GGG | 18.1(298542) |

### Plasmids:

In a second aspect, the present invention provides plasmids comprising the engineered polynucleotides described herein. As used herein, a “plasmid” is a circular double-stranded DNA molecule that can replicate independently of the genome in a cell.

As is described in greater detail in the section titled “Methods for producing an influenza virus” below, plasmid-based expression systems are commonly used to rescue infectious viruses. In such systems, a viral segment (in the form of cDNA) is inserted into a plasmid between an RNA polymerase I (pol I) promoter and a terminator sequence. This entire pol I transcription unit is flanked by an RNA polymerase II (pol II) promoter and a polyadenylation site. These plasmids comprising stacked pol I and pol II transcription units are referred to herein as “viral rescue plasmids”. The orientation of the two transcription units in the viral rescue plasmid allows for the synthesis of negative-sense viral RNA from one strand and positive-sense mRNA from the opposite strand, such that both viral RNAs and viral mRNAs/proteins are produced from the plasmid after it is transfected into a cell. Many reverse genetic systems that utilize viral rescue plasmids are known in the art, see, e.g., Neumann et al., PNAS 96:9345-9350 (1999); Fodor et al., J. Virol. 73:9679-9682 (1999); Hoffmann et al., PNAS 97:6108-6113 (2000); and Hoffmann et al., Virology 267:310-317 (2000)). In preferred embodiments, the plasmid is a pDZ plasmid used with the 8-plasmid reverse genetic system.

### Plasmid Compositions:

In a third aspect, the present invention provides plasmid compositions comprising plasmids encoding influenza virus segments 1, 2, 3, 5, 6, 7, and 8 and a plasmid described herein that encodes segment 4. Alternatively, the plasmid compositions may comprise plasmids encoding influenza virus segments 1, 2, 3, 4, 5, 7, and 8 and a plasmid described herein that encodes segment 6. Plasmids encoding the eight genome segments of influenza viruses are known in the art. For example, eight pDZ plasmids are available that each encode one of the following influenza virus segments: segment 1 (PB2), segment 2 (PB1), segment 3 (PA), segment 4 (HA), segment 5 (NP), segment 6 (NA), segment 7 (M), and segment 8 (NS).

### Cells:

In a fourth aspect, the present invention provides cells comprising a plasmid composition described herein. These cells can be used to produce influenza virus particles, as described in the section below titled “Methods for producing an influenza virus”. Thus, the present invention further provides influenza viruses produced from these cells.

### Influenza Viruses:

In a fifth aspect, the present invention provides influenza viruses comprising an engineered polynucleotide described herein. Influenza viruses are negative-sense, single-stranded RNA viruses of the Orthomyxoviridae family. Influenza viruses can be divided into four distinct subtypes (i.e., influenza A, influenza B, influenza C, and influenza D) based on their nucleoproteins and the antigen determinants of their matrix proteins. Human influenza A and B viruses are responsible for the seasonal flu. Thus, in preferred embodiments, the influenza viruses of the present invention are influenza A or influenza B.

As is noted above, the genomes of influenza viruses contain eight segments of single-stranded RNA that each encode 1-2 essential viral proteins. Thus, in addition to the engineered polynucleotide, the influenza viruses must comprise segments encoding the viral proteins PB2, PB1, PA, NP, NA, M1, M2, NS1, and NS2 to be functional. In some embodiments, the influenza virus comprises unmodified viral proteins selected from the group consisting of: PB2, PB1, PA, NP, NA, M, and NS. As used herein, an “unmodified” protein is a protein that does not include any additional amino acids at either the N-terminus or the C-terminus of the protein as compared to the native form of the protein.

As is noted above, the engineered polynucleotides of the present invention may encode a self-cleaving polypeptide between the encoded RBD-NA fusion protein and HA protein such that these proteins are co-translationally separated. This allows these proteins to be independently packaged onto viral particles. Thus, in some embodiments, the influenza virus expresses both the RBD of the SARS-CoV-2 spike protein and the influenza HA protein on its surface.

The term “viral particle” refers to the extracellular phase of a virus. An influenza viral particle consists of a nucleic acid core (i.e., the viral genome), an outer protein coating or capsid, and an outer envelope made of protein and phospholipid membrane derived from the host cell that produced the viral particle.

The influenza viruses of the present invention may be replication competent. As used herein, the term “replication competent” describes the ability of a virus to replicate in embryonated chicken eggs or cell culture. A replication competent virus need not have the ability to replicate in a host cell in vivo. To replicate, a virus must replicate its genome, synthesize all essential viral proteins, and assemble these components into viral particles. In some embodiments, the influenza virus can propagate itself in embryonated chicken eggs or in cell culture.

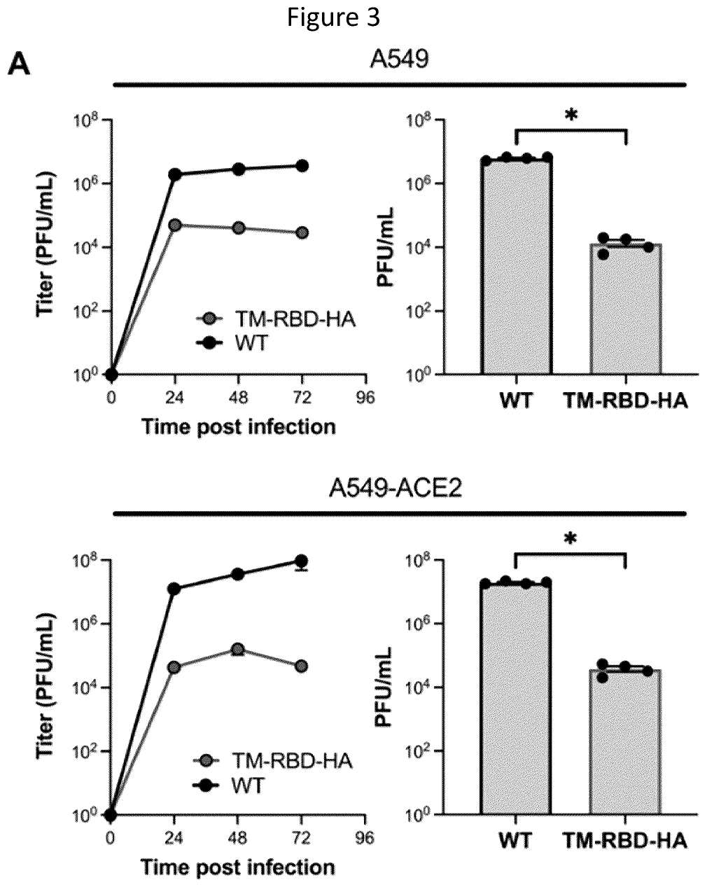

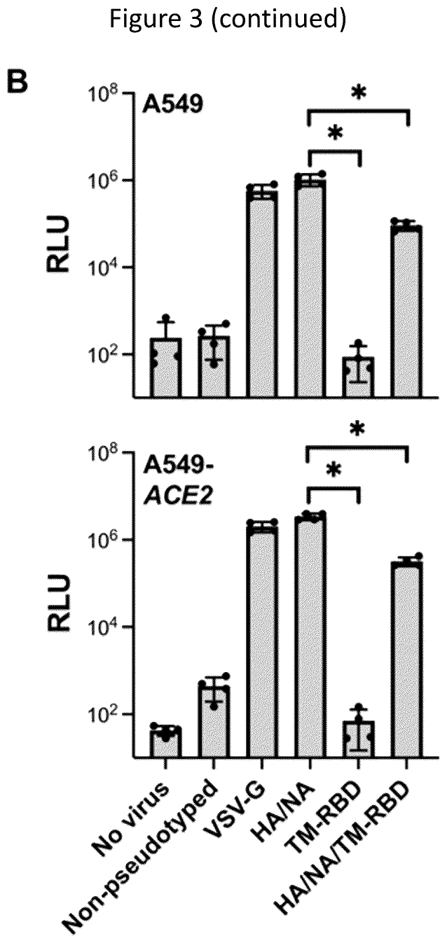

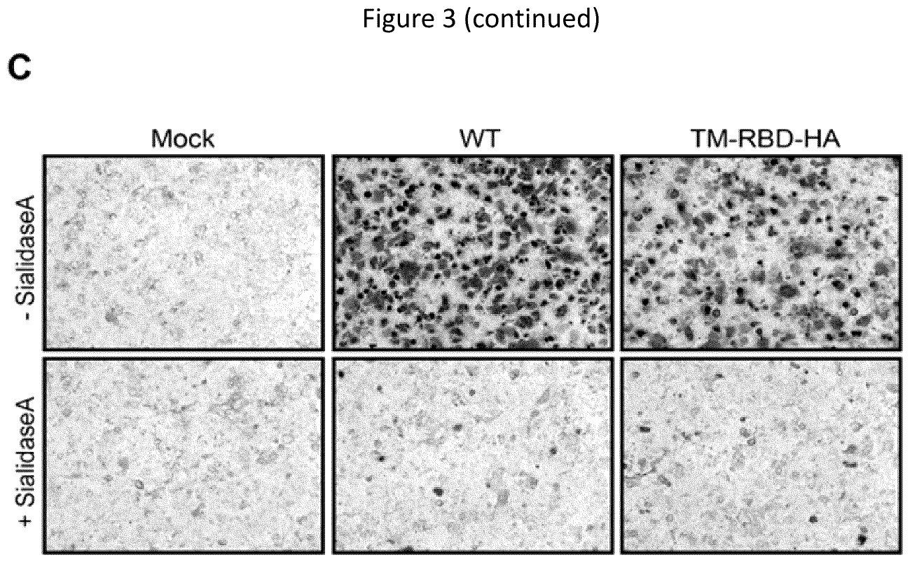

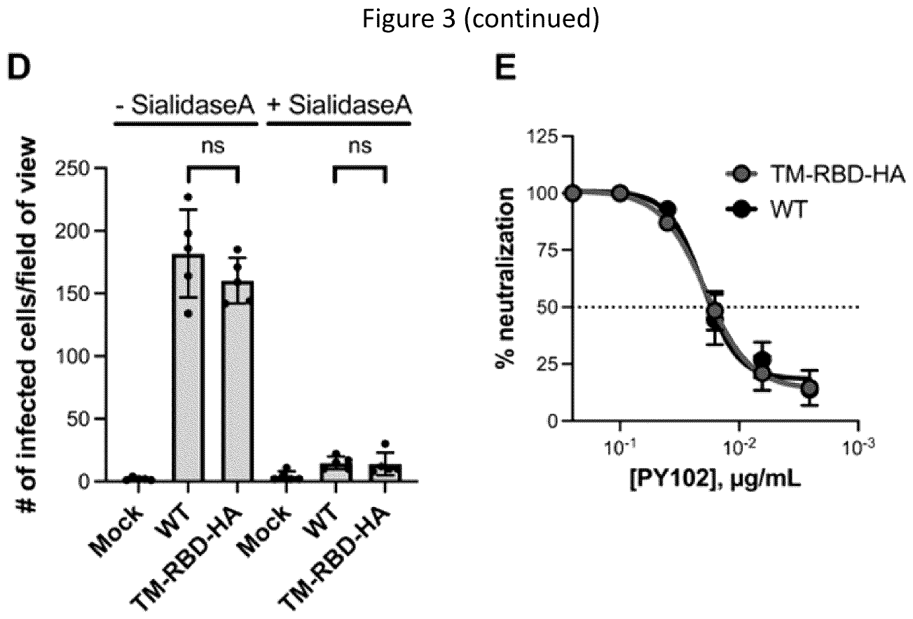

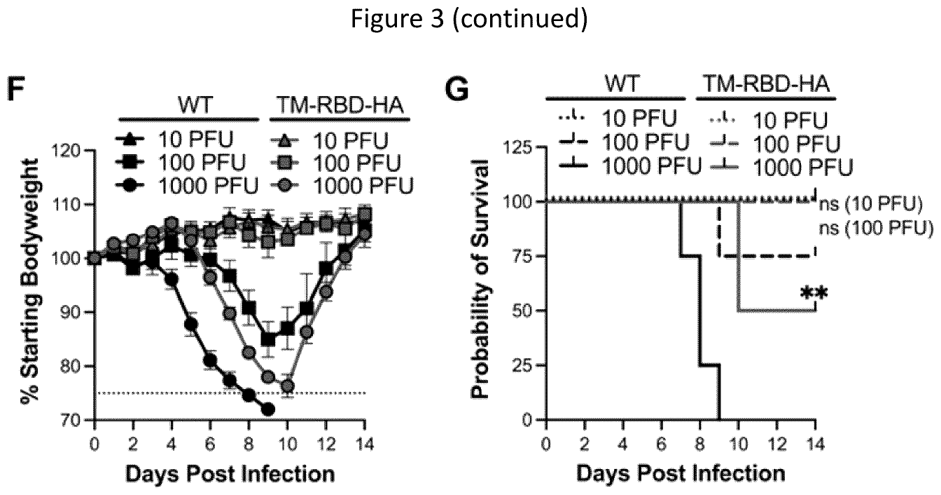

While insertion of an RBD upstream of HA in segment 4 of the IAV genome was shown to attenuate the virus tested in the Examples (FIG. 3), the influenza viruses of the present invention may be inactivated or further attenuated to make them safer for use as a vaccine. The term “inactivated” is used to describe a pathogenic virus that has been killed such that it can no longer cause disease. Viruses may be inactivated using heat, chemicals (e.g., formaldehyde, formalin, and beta-propiolactone), or radiation. The term “attenuated” is used to describe a pathogenic virus that has been weakened so that it cannot cause disease. Live attenuated viruses are often used as vaccines because they tend to stimulate a stronger and more durable immune response than inactivated viruses. Viruses may be attenuated by serial passaging the virus through a foreign host (e.g., tissue culture, embryonated chicken eggs, live animals). As the virus evolves in the new host, it will gradually lose its efficacy in the original host due to the lack of selection pressure. Viruses may also be attenuated via reverse genetics (e.g., introduction of a mutation that weakens the virus). For example, mutations that cold-adapt the virus (i.e., limit its replication above a particular temperature, thereby limiting the spread of the virus in the respiratory tract) can be introduced.

In some embodiments, the engineered influenza viruses comprise polynucleotides encoding RBDs on more than one viral segment (e.g., segment 4 and segment 6). The multiple RBDs could be from different variants of SARS-CoV-2. Thus, the ability to encode multiple RBDs in a single influenza virus would allow a single engineered virus to provide protection against multiple SARS-CoV-2 variants (e.g., the prominent SARS-CoV-2 variants that are circulating or that are projected to circulate).

### Pharmaceutical Compositions:

In a sixth aspect, the present invention provides pharmaceutical compositions comprising an influenza virus described herein and a pharmaceutically acceptable carrier. Pharmaceutically acceptable carriers are known in the art and include, but are not limited to, diluents (e.g., Tris-HCl, acetate, phosphate), preservatives (e.g., Thimerosal, benzyl alcohol, parabens), solubilizing agents (e.g., glycerol, polyethylene glycerol), emulsifiers, liposomes, and nanoparticles. Pharmaceutically acceptable carriers may be aqueous or non-aqueous solutions, suspensions, or emulsions. Examples of nonaqueous solvents are propylene glycol, polyethylene glycol, vegetable oils such as olive oil, and injectable organic esters such as ethyl oleate. Aqueous carriers include isotonic solutions, alcoholic/aqueous solutions, emulsions, or suspensions, including saline and buffered media.

The pharmaceutical compositions of the present invention may further include additives such as albumin or gelatin to prevent absorption to surfaces, detergents (e.g., Tween 20, Tween 80, Pluronic F68, bile acid salts), anti-oxidants (e.g., ascorbic acid, sodium metabisulfite), bulking substances or tonicity modifiers (e.g., lactose, mannitol). Components of the compositions may be covalently attached to polymers (e.g., polyethylene glycol), complexed with metal ions, or incorporated into or onto particulate preparations of polymeric compounds (e.g., polylactic acid, polyglycolic acid, hydrogels, etc) or onto liposomes, microemulsions, micelles, bilamellar or multilamellar vesicles, erythrocyte ghosts, or spheroplasts. The compositions may also be formulated in lipophilic depots (e.g., fatty acids, waxes, oils) for controlled or sustained release.

The pharmaceutical compositions may also include adjuvants to increase their immunogenicity. Suitable adjuvants include, without limitation, mineral salt adjuvants, gel-based adjuvants, carbohydrate adjuvants, cytokines, or other immunostimulatory molecules. Exemplary mineral salt adjuvants include aluminum adjuvants, salts of calcium (e.g. calcium phosphate), iron, and zirconium. Exemplary gel-based adjuvants include aluminum gel-based adjuvants and acemannan. Exemplary carbohydrate adjuvants include inulin-derived adjuvants (e.g., gamma inulin, algammulin) and polysaccharides based on glucose and mannose (e.g., glucans, dextrans, lentinans, glucomannans, galactomannans). Exemplary cytokines include IFN-γ, granulocyte-macrophage colony stimulating factor (GM-CSF), IL-2, and IL-12. Suitable adjuvants also include any FDA-approved adjuvants for influenza vaccine usage including, without limitation, aluminum salt (alum) and the squalene oil-in-water emulsion systems MF59 (Wadman 2005 (Novartis)) and AS03 (GlaxoSmithKline).

In some embodiments, the pharmaceutical compositions include a concentration of total non-infectious viral particles of at least 10⁶ pfu/mL, at least 10⁷ pfu/mL, at least 10⁸ pfu/mL, at least 10⁹ pfu/mL, at least 10¹⁰ pfu/mL, or at least 10¹¹ pfu/mL.

### Methods for Generating an Immune Response:

In a seventh aspect, the present invention provides methods for generating an immune response to both influenza and SARS-CoV-2 in a subject. The methods comprise administering a therapeutically effective amount of an influenza virus or pharmaceutical composition described herein to the subject. In these methods, the influenza virus or pharmaceutical composition serves as a vaccine. A “vaccine” is a composition comprising an antigen that is administered to a subject to stimulate an immune response to the antigen in the subject. As used herein, the term “antigen” refers to a molecule that can initiate a humoral and/or a cellular immune response in a recipient.

An “immune response” is the reaction of the body to the presence of a foreign substance (i.e., an antigen). The immune response induced by the present methods may comprise a humoral immune response (e.g., a B-cell or antibody response), a cell-mediated immune response (e.g., a T-cell immune response), or both a humoral and cell-mediated immune response. The immune response can include, for example, the production of antibodies against the HA protein, the RBD, or both the HA protein and the RBD. The immune response of a subject to the vaccine may be evaluated through measurement of antibody titers, neutralizing antibody response or lymphocyte proliferation assays, or by monitoring signs and symptoms after challenge with the corresponding pathogen, such as weight loss, morbidity or mortality. The protective immunity conferred by the present methods may be evaluated by measuring a reduction in clinical signs, e.g., the mortality, morbidity, temperature, physical condition, or overall health of the subject.

As used herein, the term “administering” refers to the introduction of a substance into a subject's body. Common methods of administering a vaccine include oral administration, subcutaneous administration, intramuscular administration, intradermal administration, and intranasal administration. The vaccines can be administered as a single dose or in several doses. For example, the vaccines may be administered two or more times separated by 4 hours, 6 hours, 8 hours, 12 hours, a day, two days, three days, four days, one week, two weeks, or by three or more weeks.

The term “therapeutically effective amount” refers to an amount of a vaccine that is sufficient to induce an immune response in a subject receiving the vaccine. The therapeutically effective amount will vary depending on the formulation of the vaccine, the influenza and its severity, and the age, weight, physical condition, and responsiveness of the subject. Typical therapeutically effective amounts include 5 mg, 10 mg, 15 mg, 20 mg, 25 mg, 30 mg, 35 mg, 40 mg, 45 mg, 50 mg, 55 mg, 60 mg, 65 mg, 70 mg, 75 mg or more of HA per vaccine virus strain per 0.5 mL dose.

In some embodiments, the methods prevent or reduce the symptoms of influenza and/or COVID19 in the subject. The symptoms of influenza include, but are not limited to, headaches, chest discomfort, cough, sore throat, fever, aches, chills, fatigue, weakness, sneezing, and stuffy nose. The symptoms of COVID19 include, but are not limited to, fever, chills, cough, shortness of breath or difficulty breathing, fatigue, muscle or body aches, headaches, new loss of taste or smell, sore throat, congestion or runny nose, nausea or vomiting, and diarrhea.

The “subject” to which the methods are applied may be a mammal or a non-mammalian animal, such as a bird. Suitable mammals include, but are not limited to, humans, cows, horses, sheep, pigs, goats, rabbits, dogs, cats, bats, mice, and rats. In certain embodiments, the methods may be performed on lab animals (e.g., mice and rats) for research purposes. In other embodiments, the methods are used to treat commercially important farm animals (e.g., cows, horses, pigs, rabbits, goats, sheep, and chickens) or companion animals (e.g., cats and dogs). In a preferred embodiment, the subject is a human.

The influenza viruses described herein may be administered alone or in combination with additional influenza viruses. For example, a quadrivalent IAV vaccine could include RBDs from up to four strains of SARS-CoV-2, allowing it to provide broad protection against circulating SARS-CoV-2 variants.

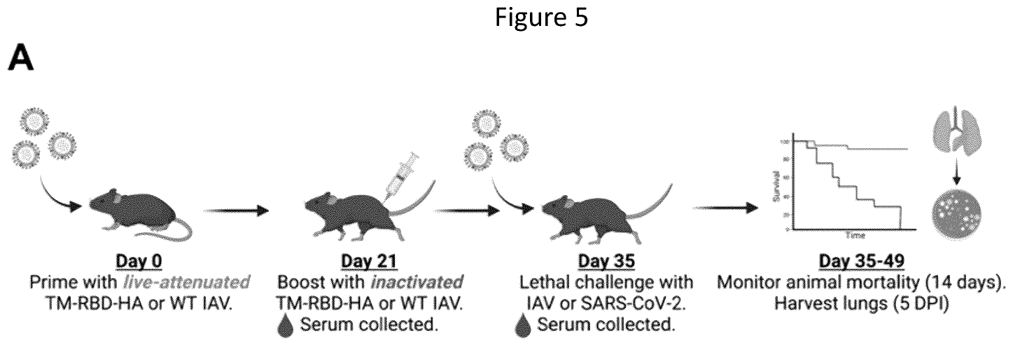

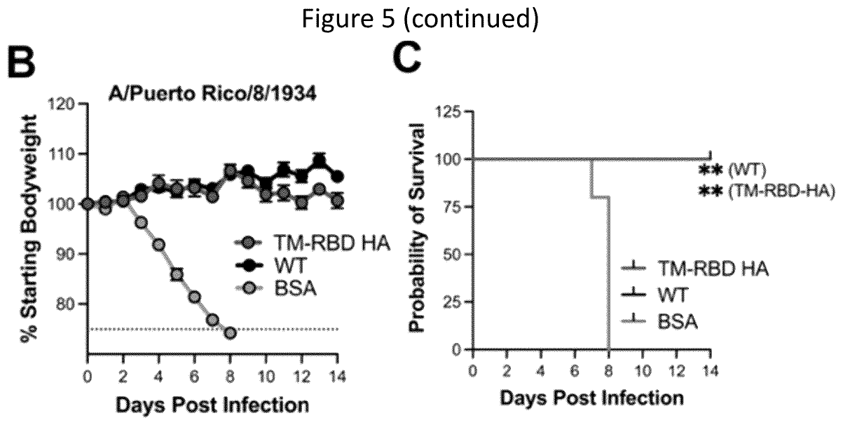

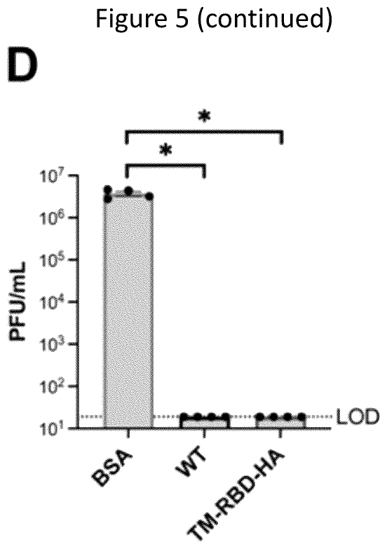

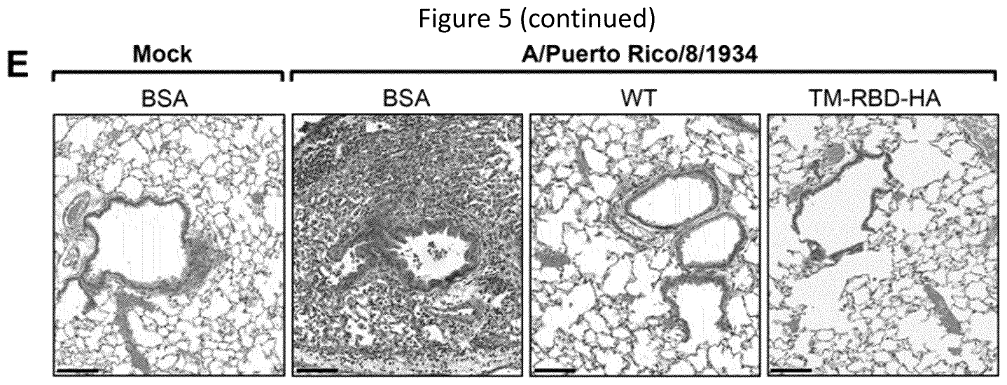

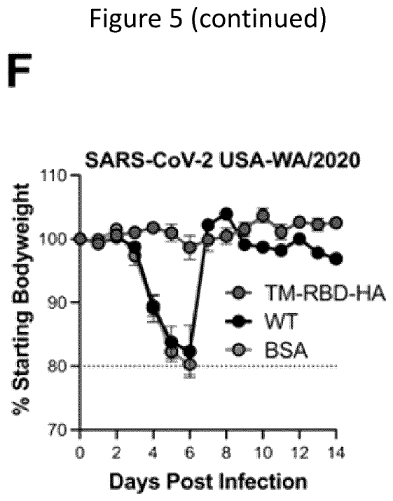

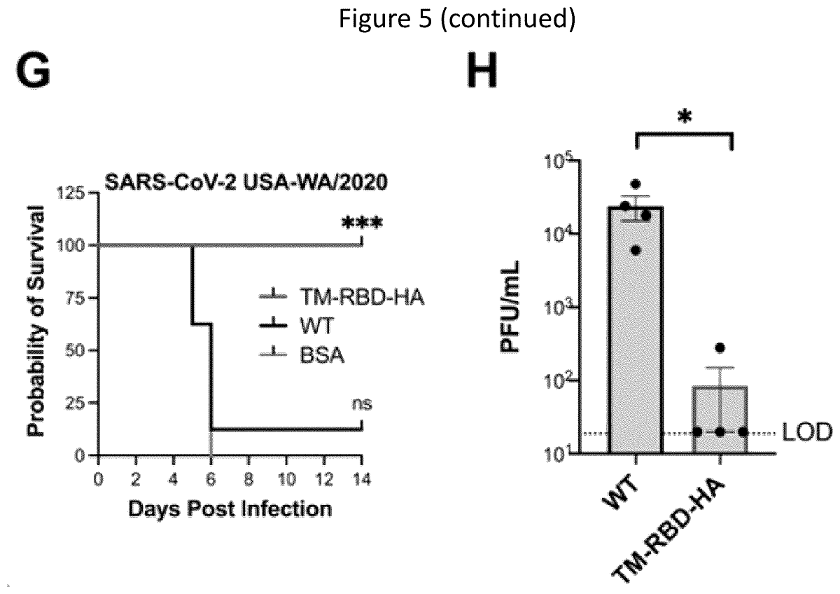

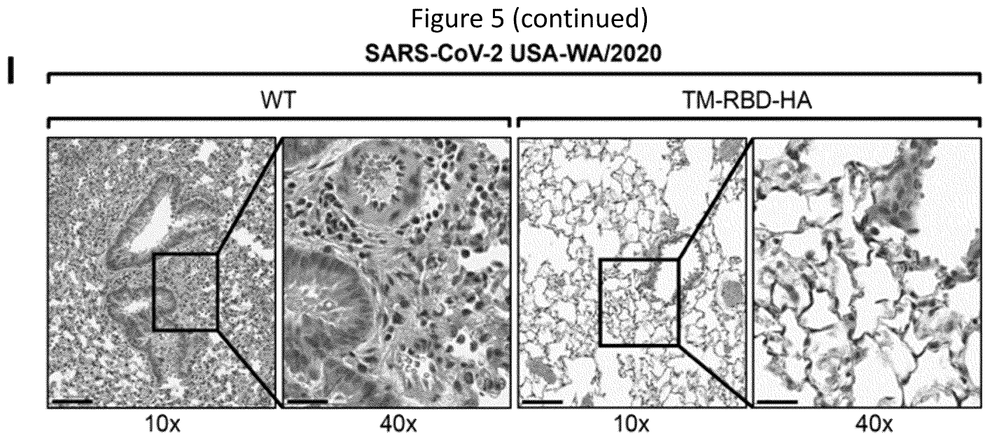

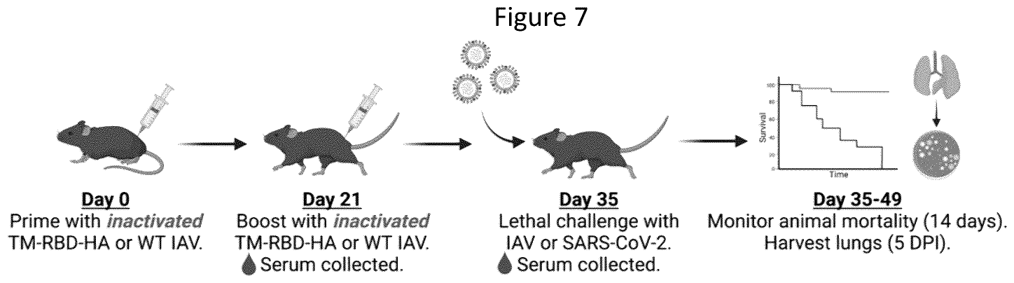

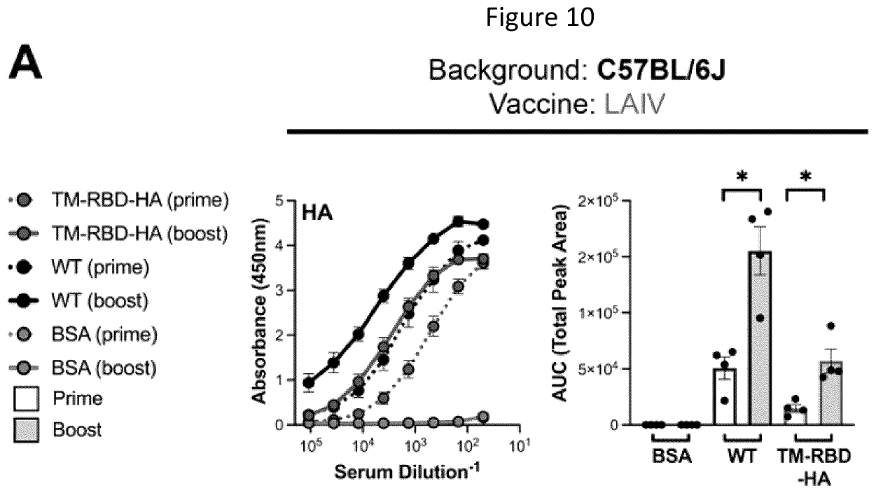

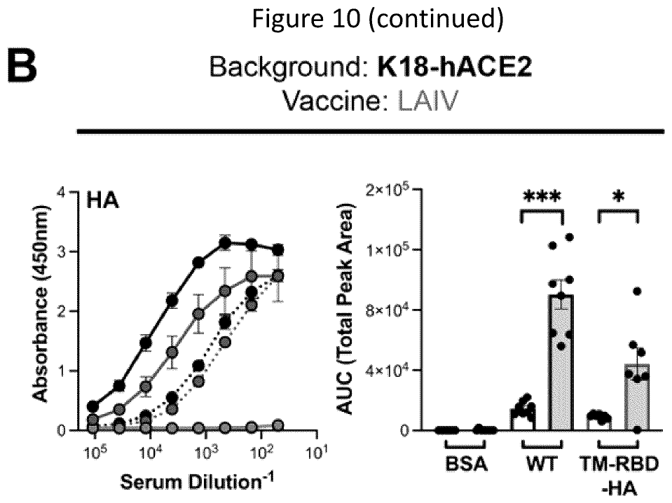

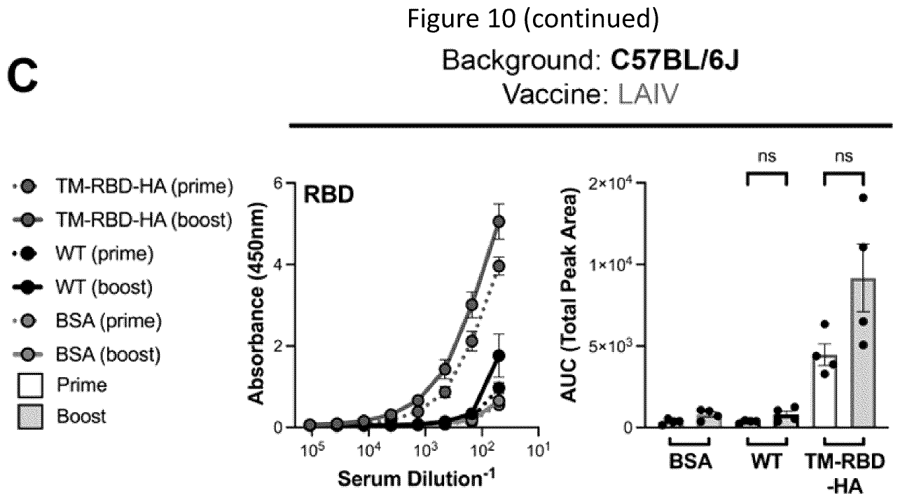

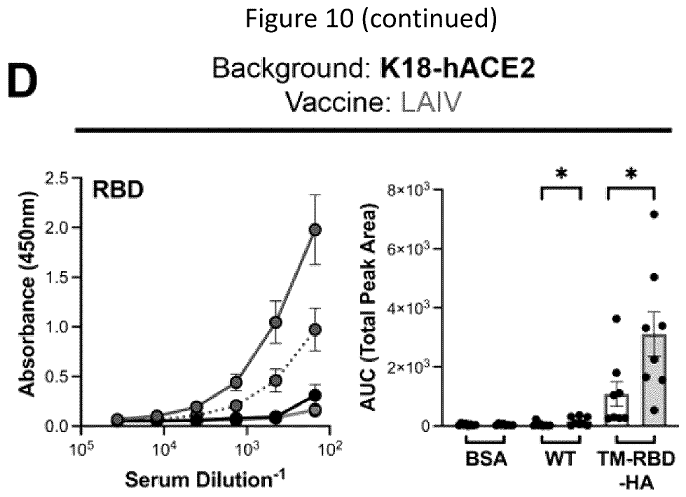

In the Examples, the inventors tested their engineered influenza virus in two different prime-boost vaccination schemes. Thus, in some embodiments, the influenza virus or pharmaceutical composition is administered in a prime-boost vaccination scheme (i.e., a scheme in which a vaccine is given at two different timepoints). The inventors found that when used in a prime-boost vaccination scheme in which both the prime and the boost comprised inactivated virus, their engineered viruses were unable to generate sufficient antibodies against the RBD to protect mice against a lethal SARS-CoV-2 challenge (FIGS. 7-9). However, they found that when they were used in a prime-boost scheme in which the prime was live-attenuated virus and the boost was inactivated influenza virus, their engineered viruses provided protection against SARS-CoV-2 (FIG. 5). When both the prime and the boost were with the live-attenuated influenza virus, protection was provided against both influenza and SARS-CoV-2 as well (FIG. 10). Thus, in some embodiments, the prime comprises live-attenuated influenza virus and the boost comprises inactivated or live-attenuated influenza virus.

### Methods for Producing an Influenza Virus:

In an eighth aspect, the present invention provides methods for producing an influenza virus. The methods comprise rescuing the virus with a plasmid composition described herein. “Virus rescue” is a technique that is used to produce recombinant viruses. In this technique, each segment of the viral genome is cloned into a viral rescue plasmid in the form of cDNA. Specifically, the viral segment is cloned into a pol I transcription unit that is flanked by a pol II transcription unit in the viral rescue plasmid. Plasmids encoding each segment of the viral genome are transfected into a cell. In the cell, the plasmids are transcribed to produce negative-sense viral RNA from one strand and positive-sense mRNA from the opposite strand, such that all viral RNAs and mRNAs/proteins are expressed and packaged into viral particles (see, e.g., PNAS 99 (17) 11411-11416, 2002).

Thus, the methods of making a virus may comprise (a) transfecting a plasmid composition described herein into a cell; (b) incubating the transfected cell; and (c) harvesting the influenza virus produced by the cell. These methods produce influenza viruses that express both the RBD of the SARS-CoV-2 spike protein and the influenza hemagglutinin protein on their surface. As used herein, the term “transfecting” refers to a process of artificially introducing polynucleotides into cells. Transfection may be performed under natural or artificial conditions. Suitable transfection methods include, without limitation, lipofection, bacteriophage or viral infection, electroporation, heat shock, microinjection, and particle bombardment.

The cell lines that are transfected with the viral rescue plasmids in the present methods are eukaryotic cell lines. Suitable eukaryotic cells include, without limitation, mammalian cells or chicken cells. The cell may be a cell in culture or may be an embryonated chicken egg. Suitable mammalian cells include, without limitation, MDCK cells, A549 cells, CHO cells, HEK293 cells, HEK293T cells, HeLa cells, NS0 cells, Sp2/0 cells, COS cells, BK cells, NIH3T3 cells, FRhL-2 cells, MRC-5 cells, WI-38 cells, CEF cells, CEK cells, DF-1 cells, and Vero cells.

In embodiments in which the cell is a cultured cell, the virus may be harvested by collecting the supernatant from the culture by, for example, via centrifugation or pipetting. In embodiments in which the cell is an embryonated chicken egg, the virus may be harvested by collecting the allantoic fluid from the embryonated chicken egg.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

The following examples are meant only to be illustrative and are not meant as limitations on the scope of the invention or of the appended claims.

### Examples

Vaccines targeting SARS-CoV-2 have been shown to be highly effective, but the breadth required to protect against emerging variants and the longevity of the protection provided by these vaccines remain unclear. Post-immunization boosting has been shown to be beneficial for disease protection and, as new variants continue to emerge, periodic (and perhaps annual) vaccination will likely be recommended. New seasonal influenza virus vaccines currently need to be developed every year due to continual antigenic drift, an undertaking made possible by a robust global vaccine production and distribution infrastructure.

To create a seasonal combination vaccine targeting both influenza viruses and SARS-CoV-2 that is also amenable to frequent reformulation, the present inventors have developed an influenza A virus (IAV) genetic platform that allows immunogenic domains of the SARS-CoV-2 spike (S) protein to be incorporated onto IAV particles. In the following example, the inventors demonstrate that vaccination with this combination vaccine elicits neutralizing antibodies and provides protection against lethal challenge with both pathogens in mice. This approach may allow the established influenza vaccine infrastructure to be leveraged to generate a cost-effective and scalable seasonal vaccine for both influenza and coronaviruses.

### Materials and Methods

### Cell Lines and Viruses

Human embryonic kidney cells (HEK293T, ATCC) were grown in Dulbecco's Modified Eagle Medium supplemented with 5% fetal bovine serum, GlutaMAX (Gibco cat. no. 35050079), and penicillin/streptomycin. Madin-Darby canine kidney cells (MDCK, ATCC) were grown in minimum essential medium (MEM) supplemented with 5% fetal bovine serum (FBS), GlutaMAX, HEPES, NaHCO₃, and penicillin/streptomycin. All cells were grown at 37° C. under 5% CO₂. African green monkey kidney cells (Vero E6, ATCC) were grown in MEM+Earl's Salts+L-Glutamine (Gibco 11095-080). This media was supplemented with penicillin/streptomycin, 10% FBS, 1 mM sodium pyruvate, and 1×MEM NEAA (Gibco 11140-050). ST cells (ATCC CRL-1746) were grown in EMEM supplemented with penicillin/streptomycin and 10% FBS. A/Puerto Rico/8/1934 (PR8) virus was used for recombinant virus generation as well as vaccination/animal challenge experiments. SARS-related coronavirus 2, isolate USA-WA1/2020, NR-52281 was used for SARS-CoV-2 infections and is from BEI Resources. To account for mutations in viral genomes during propagation, each genomic segment of all strains used for challenge in this study was subjected to Sanger sequencing. GenBank accession numbers for the A/Puerto Rico/8/1934 viral genes (with mutations noted in parentheses) are as follows: PB2, AF389115.1; PB1, CY148249.1 (A549C→K175N); PA, AF389117.1 (A1025T→Y334F); HA, AF389118.1; NP, AF389119.1; NA, AF389120.1; M, AF389121.1; and NS, AF389122.1. GenBank accession numbers and mutations for the TM-RBD-HA vaccine virus are as follows: PB2, AF389115.1; PB1, CY148249.1 (A549C→K175N); PA, AF389117.1 (A1025T→Y334F); HA, AF389118.1 (G1456A→E451K, A1683G→1526M); NP, AF389119.1 (C515T→T1571); NA, AF389120.1; M, AF389121.1 (T1027A→silent); and NS, AF389122.1.

### Cloning and Rescue of A/Puerto Rico/8/1934 Encoding SARS-CoV-2 RBD

Influenza segment cloning was accomplished as previously described²⁹. First, the RBD (i.e., amino acids 319-541 of the spike protein) from the SARS-CoV-2 Wuhan-Hu-1 isolate (Accession: MN908947.3) was codon optimized for expression in influenza A viruses and was synthesized by IDT. Importantly, we also encoded the NA transmembrane domain (i.e., amino acids 1-40 of the NA protein) 5′ to the RBD to allow it to be incorporated into the viral particle, and encoded a FLAG tag 3′ to the RBD to aid in its detection. The codon optimized RBD was PCR amplified and cloned into the bicistronic pDZ rescue plasmid system for A/Puerto Rico/8/1934 using the NEBuider HiFi DNA assembly kit (NEB). Specifically, the SARS-CoV-2 TM-RBD construct was cloned into the previously reported mNeon-HA construct, wherein the TM-RBD sequence replaced the mNeon reporter, allowing for expression of the transgene 5′ to HA²⁹. Successful cloning was confirmed by Sanger sequencing. Viral rescue was then performed by transfecting the TM-RBD-HA plasmid along with seven plasmids encoding the other PR8 segments into 293T cells using the Mirus TransIT-LT1 reagent. Rescued virus was then amplified via inoculation into 10-day-old embryonated chicken eggs (Charles River) at 37° C. for three days. The resulting plaque was then purified and each individual plaque was amplified again in 10-day-old embryonated chicken eggs.

### Virus Propagation and Growth Kinetics

Influenza virus titering was performed as previously reported²⁹. Briefly, approximately 500 PFU of each plaque-purified stock was injected in 10-day old embryonated hen eggs purchased from Charles River Laboratories, Inc. and incubated for 72 hours at 37° C. The allantoic fluid was then harvested and the titer was determined via plaque assay on MDCK cells. This was accomplished by serially diluting allantoic fluid and then incubating MDCKs with 500 μL of the diluted sample for 1 hour at 37° C. After incubation, the viral suspension was aspirated, agar overlay was applied, and cells were incubated at 37° C. for 48-72 hours depending on plaque size. Plaque assays were then fixed by adding 2 mL of 4% formaldehyde solution and incubating overnight at room temperature. The next day, formaldehyde was aspirated, and cells were washed prior to performing antigen staining to detect A/Puerto Rico/8/1934 HA protein. For antigen staining, fixed monolayers were stained with a 1:2000 dilution of vaccinated mouse serum in antibody dilution buffer (5% milk, 0.05% Tween-20 in PBS). Plaque assays were incubated at 4° C. for 2 hours/overnight and then stained with a 1:4000 dilution of HRP-conjugated anti-mouse secondary antibody (Novex cat no. NXA931V) for 2 hours at room temperature. Plaque assays were developed with 0.5 mL of the True Blue peroxidase substrate (KPL). For viral growth curves and endpoint titer assays, eggs were injected with 10,000 PFU/egg and eggs were collected for plaque assay at 24, 48, and 72 hours post-infection. Hemagglutination assays were performed by serially diluting virus 1:2 in PBS in a 96-well plate then adding chicken blood to each well. Plates were incubated for 1 hour and then each well was scored as positive or negative.

SARS-CoV-2 stocks for in vitro assays were grown on Vero E6 cells in virus infection media (MEM+Earl's Salts, penicillin/streptomycin, 2% FBS, 1 mM sodium pyruvate, 1×MEM NEAA) for 72 hours. Stocks were frozen at −80° C. and were titered by serially diluting virus in virus infection media and then infecting a confluent monolayer of Vero E6 cells growing in 6-well, poly-L-lysine treated plates for 1 hour. Inoculum was then removed, and an agarose overlay was added. Cells were incubated at 37° C. and 5% CO₂ for 72 hours and were then stained with 0.05% neutral red in PBS for 3 hours. SARS-CoV-2 stocks for animal infections were grown on ST cells in virus infection media. Virus was titered using a similar protocol as above but using a methylcellulose overlay. The virus was incubated for 4 days and the monolayers were stained with crystal violet.

### Viral Purification

Purification of influenza viral particles was performed prior to use in vaccination and ELISA experiments. First, viral stocks were grown in 10-day-old embryonated hen eggs as described above. Then allantoic fluid was collected and dialyzed overnight using the Spectra-Por Float-a-lyzer G2 10 mL, 100 kDa MWCO tubes according to manufacturer's instructions (Millipore Sigma cat. no. Z727253-12EA). After the virus samples were dialyzed to remove larger impurities, the allantoic fluid was collected and the samples were concentrated by ultracentrifugation using a 30% sucrose cushion for 1 hour at 25,700 RPM using the Sorvall TH-641 swinging bucket rotor. Virus samples were then resuspended in PBS and pooled prior to being fixed in 0.02% formalin for 30 minutes at room temperature. Samples were then once again dialyzed overnight to remove formalin using Slide-A-Lyzer cassettes (Thermo Scientific cat. no. PI66370) before being stored at 4° C. until use.

### RT-PCR and Serial Passage Experiments

Viral RNA was extracted using TRIzol (Invitrogen) followed by chloroform/ethanol precipitation or using a Qiagen viral RNA miniprep kit. RT-PCR reactions were performed using a SuperScript™ III One-Step RT-PCR System kit (Thermo cat. no. 12574026) according to the manufacturer's guidelines. The following primers were used: HA 5′ Forward: 5′-GTAGATGCAGCAAAAGCAGGGGAAAATA-3′ (SEQ ID NO: 16), HA 3′ Reverse: 5′-CCATCCTCAATTTGGCAC-3′ (SEQ ID NO: 17), RC093: 5′-AGCAAAAGCAGGGGAAAATA-3′ (SEQ ID NO: 18), and RC095: 5′-GTCTTCGAGCAGGTTAACAG-3′ (SEQ ID NO: 19). RT-PCR reactions using RNA generated from miniprep kits varied in template concentration, as the presence of carrier RNA inhibits nucleic acid quantification via photometric means. The RT-PCR products were analyzed on 0.8-1% agarose gels run at 50 V. For serial passage experiments, an 80% confluent monolayer of MDCK cells was infected with a MOI of 0.01 for 48 hours. After 48 hours, cell supernatants were collected and centrifuged for 5 minutes at 500×g. Supernatant was removed and 100 μl was added to 1 mL of Trizol and frozen at −80° C. The titer of the remaining virus was then estimated via hemagglutination assay and a new monolayer of cells was subsequently infected. This protocol was repeated 10 times.

### Microscopy

MDCK cells were seeded in 24-well plates containing glass coverslips coated with poly-L-lysine and allowed to grow at 37° C. for 24 hours prior to infection. Cells were washed with PBS and infected with either PR8 or TM-RBD-HA virus at an MOI of 0.25 in infection media (1 mM KH₂PO₄, 155 mM NaCl, Na₂HPO₄, 83.5 mM CaCl₂), 105 mM MgCl₂, 10 U/mL penicillin/streptomycin, 0.4% BSA) for 1 hour at 37° C. Virus was removed from cells and replaced with post-infection media (Gibco OptiMem supplemented with 0.01% FBS, 10 U/mL penicillin/streptomycin, 0.4% BSA, and 1 μg/mL TCPK-trypsin). Cells were incubated for 24 hours and fixed with methanol-free 4% formaldehyde (Fischer cat. no. PI28906). DNA was visualized using Hoechst 33342 (Thermo) at 5 μg/mL in PBS for 15 minutes. HA and SARS-CoV-2 were detected using PY102 and SARS-CoV-2 Spike Protein (RBD) Polyclonal Antibody (Thermo cat. no. PA5-114451), respectively, at a 1:250 dilution for approximately 3 hours. Primary antibodies were visualized using AlexFluor594-conjugated anti-mouse (for HA) and AlexFluor488-conjugated anti-rabbit (for RBD) secondary antibodies (Thermo cat. no. A-11032/A-11008) at a dilution of 1:500 for 1 hour at room temperature. Cells were imaged on a ZOE (BioRad) microscope.

### Western Blots

Protein extracts were quantified and normalized via Bradford assay. SDS-PAGE was performed using 4-20% polyacrylamide gels (BioRad) electrophoresed at 120 V for 60 minutes. Proteins were transferred to 0.45 μm nitrocellulose membranes at 90 V for 60 minutes at 4° C. and blocked using PBST+5% milk for a minimum of 1 hour at room temperature. For cellular lysates, 20 μg of total protein was loaded per sample. To normalize viral protein extracts, 0.5 μg of PR8 and PR8-TM-RBD HA were initially loaded and analyzed via western blot. Viral protein extracts were probed for M1 and normalized via densitometry (ImageJ, NIH). After normalization to M1, 0.5 μg PR8 and 1.32 μg PR8-TM-RBD HA were loaded for subsequent western blot analyses. The following antibodies were used for protein detection: PY102 (HA, 1 μg/mL), 4A5 (NA, 0.45 μg/mL), anti-matrix protein [E10](M1 and M2, 1:1,000, Kerafast cat. no. EMS009), and anti-SARS-CoV-2 spike protein (RBD) polyclonal antibody (RBD, 1:1,000, Invitrogen cat. no. PA5-114451). All primary antibodies were diluted in PBST+5% milk and applied to membranes for at least 16 hours at 4° C. Anti-mouse (1:20,000, Thermo cat. no. A16072) and anti-rabbit (1:10,000, Thermo cat. no. A16104) secondary antibodies were diluted in PBST+5% milk and applied to membranes for 60 minutes at room temperature. Membranes were developed using Clarity ECL or Clarity ECL MAX.

### Purification of Soluble HA and RBD Proteins for ELISAs

6×-His-tagged proteins were overexpressed in Expi293F cells (Thermo cat. no. A14527) via transfection with expression vectors. Cells were lysed via sonication and clarified lysates were applied to Ni-NTA columns. Eluted protein fractions were combined, dialyzed, and stored at −80° C.

### ELISAs

Proteins/viruses were immobilized to 96-well plates using carbonate coating buffer (30 mM Na₂CO₃, 70 mM NaHCO₃, pH 9.5) for at least 16 hours at 4° C. For protein samples, 50-100 μL of sample at 10 μg/mL was added to wells. For viral samples, 1×10⁶ PFUs were added to wells in a volume of 50-100 μL. All samples were diluted using PBS+3% BSA. After coating, wells were washed with PBS and blocked with PBS+3% BSA for at least 1 hour at room temperature. Primary antibodies were diluted using PBS+3% BSA and incubated with immobilized proteins/viruses for at least 1 hour at room temperature (anti-RBD ProSci cat. no. 9087, anti-HA PY102 a kind gift from Dr. Tom Moran (Icahn School of Medicine at Mount Sinai), DH1041 and DH1044 kind gifts from Drs. Bart Haynes, Kevin Saunders, and Dapeng Li (Duke)). Anti-human (1:10,000 Thermo cat. no. A18805), anti-mouse (1:5,000, Thermo cat. no. A16072), and anti-rabbit (1:5,000, Thermo cat. no. A16104) secondary antibodies were diluted in PBS+3% BSA and incubated with immobilized proteins/virus for 1 hour at room temperature. ELISAs were developed using 1-Step Ultra TMB-ELISA substrate (Thermo cat. no. 34029) and quenched with 2M H₂SO₄.

### Animal Infections Live-Attenuated Vaccination

Animal infections were performed using age-matched C57BL/6J (Jackson Labs 000664) or B6.Cg-Tg(K18-ACE2)2Prlmn/J mice (Jackson Labs 034860). For influenza infections, mice were anesthetized using 100 μL of a 14.2 mg/mL ketamine-xylazine mixture via intraperitoneal injection. After administration of anesthesia, mouse tails were marked or mice were injected subcutaneously with IPT-300 transponders capable of reading body temperature and animal ID (BMDS IPT-300) and baseline weights and temperatures were measured. For FIG. 3F-G, mice were inoculated with 40 μL of the indicated dose of virus diluted in pharmaceutical grade PBS. Body weight was measured daily, and mice were euthanized if their body weight reached 75% of baseline. For vaccinations using inactivated virus, 10 μg virus or BSA was injected via IM route. For LAIV vaccinations, mice were given a 40 μL intranasal inoculum of virus or PBS control, either 10-15 PFU WT A/Puerto Rico/8/1934, 100-250 PFU TM-RBD-HA, or 40 μL PBS diluted in pharmaceutical-grade PBS. Vaccination doses varied depending on which stock was used. For mice receiving a boost, another 10 μg of inactivated virus or BSA control was administered via the IM route. For challenge experiments, mice were inoculated with 500 PFUs of PR8. After infection, mice were weighed daily and euthanized if their body weight reached 75% of the baseline weight (i.e., the weight prior to infection). Euthanasia was performed via CO₂ asphyxiation as a primary method and bilateral thoracotomy as a secondary confirmation. These methods were used during both challenge experiments and administration of the live virus vaccine prime. For SARS-CoV-2 challenge experiments, prior to infection, mice were injected subcutaneously with IPT-300 transponders and baseline weights and temperatures were measured. On the day of infection, mice were anesthetized using isoflurane and then given a 50 μL intranasal inoculum of virus, 3×10⁴ PFU, diluted in pharmaceutical-grade PBS. After infection, mice were monitored daily for weight, temperature, and clinical signs (temperature and clinical signs are not reported). Mice were euthanized via CO₂ asphyxiation and bilateral thoracotomy as a secondary confirmation when their body weight reached 80% of the baseline weight measured prior to infection or they reached a clinical score of 4 in accordance with approved protocol A081-20-04. Occasionally mice did not recover from anesthesia, and these animals were excluded from subsequent experimentation and analysis.

Vaccination of Mice with Inactivated Virus

Vaccine doses for the boost were prepared using the purified inactivated virus described above. Virus samples were then diluted 1:1 with the adjuvant Addavax (Invivogen cat. no. vac-adx-10) to a final concentration of 100 μg/mL. After preparation of doses, mice were anesthetized as described above and then administered a 100 μL injection intramuscularly into the left leg. Mice were monitored the next day for side effects and were then housed for the indicated period of time before collection or viral challenge. If serum was collected, mice were anesthetized and then blood was harvested either by cheek bleed or terminal bleed. Serum was collected using Sarstedt Z-Gel tubes according to manufacturer's instruction (Sarstedt cat. no. 41.1378.005) and was then stored at −80° C. until use.

### Histological Analysis and Viral Burden Quantification in Mouse Lungs

To quantify the PFU of influenza virus or SARS-CoV-2 in the lungs of mice, lungs were collected 5 days post-infection. Lungs were placed in PBS and homogenized using Benchmark BeadBlaster 24 Microtube Homogenizer or Benchmark BeadBug Homogenizer. Homogenates were centrifuged and supernatants were removed and frozen at −80° C. PFU were quantified by diluting homogenates and performing plaque assays as described above. For histology experiments, mice were anesthetized with 200 μL of ketamine-xylazine prior to cervical dislocation. The lungs were inflated with 1.5 mL of 4% formaldehyde solution and the trachea was tied off with suture string before removal. The inflated lungs were stored in 4% formaldehyde at room temperature until they were processed for histology. Histology was performed by HistoWiz Inc. (histowiz.com) using a Standard Operating Procedure and fully automated workflow. Samples were processed, embedded in paraffin, and sectioned at 4 m. Staining was performed on a Bond Rx autostainer (Leica Biosystems) using standard protocols. Whole slide scanning was performed on an Aperio AT2 (Leica Biosystems). Images shown are at 10× and 40× magnification.

### Pseudotyped Lentivirus Transduction Experiments

The SARS-CoV-2 RBD ORF was cloned into the pCAGGS expression plasmid. To package lentivirus particles, combinations of pCAGGS-RBD, pNL4-3.Luc, pA8.74, pDZ-HA, and pDZ-NA were transfected into 293T cells using PEI and incubated at 37° C. for 72 hours. For viral particles pseudotyped with HA/NA, 293 Ts were grown in serum-free OptiMEM growth medium. Pseudotyped viruses were harvested and stored at −80° C. For transduction experiments, A549 and A549-ACE2 cells were plated in 24-well plates. To transduce cells, 100 μL of media containing pseudotyped viruses was added to each culture and incubated at 37° C. for 48 hours. Transduction efficiency was determined via luciferase assays using a firefly luciferase detection kit (Promega cat. no E1500). Luciferase intensities were measured using a luminometer. pNL4-3.Luc and pA8.74 plasmids were a kind gift from Dr. Bryan Cullen (Duke University).

### SialidaseA Treatment of Cells

SialidaseA (Agilent cat. no. GK80040) was used to cleave α2,3- and α2,6 N-linked sialic acids from the surfaces of cells. Briefly, growth medium supplemented with SialidaseA at 30 mU/mL was exchanged for the medium on cells. Cells were incubated with SialidaseA-containing medium for 3-5 hours at 37° C. After incubation, media was removed, and cells were washed with PBS to remove residual SialidaseA. Cells were subsequently infected with either WT or TM-RBD-HA virus (MOI=1) for 1 hour. Cells were grown in complete growth medium and fixed with 4% paraformaldehyde after 24 hours. Virus-infected cells were detected using PY102 antibody (1:2,000), stained using True Blue peroxidase substrate (KPL), and imaged on a ZOE microscope (BioRad). The number of infected cells per field of view was determined using FIJI (NIH); image threshold values were set to 0-85 and particles (size: 1,000-1,000,000 px, circularity: 0-infinity) were counted. Five non-overlapping areas of each well were imaged and used for quantification.

### Plaque Reduction Neutralization Assays

MDCK and Vero cells were used for influenza and SARS-CoV-2 plaque reduction assays, respectively. A master mix of virus was diluted to the indicated concentration (˜40-80 PFU/mL) and aliquoted prior to being mixed with antibody dilutions (from sera or purified monoclonal antibodies). Following a 45-minute incubation at room temperature with antibody, the media was aspirated from cells and 500 μL of the virus-antibody mixture was added to each well of cells. For each experiment a no antibody control was included to accurately record how much virus had been used to infect cells. Cells were incubated with the virus-antibody mixture at 37° C. for 1 hour, rocking the samples every 15 minutes to ensure that the cells were completely covered by the solution. After this period, the solution was aspirated, and an agar overlay was applied. For influenza plaque reduction assays, staining and plaque counting was performed as described above in the titering section. SARS-CoV-2 plaque reduction assays were evaluated by first staining plaques with 0.05% Neutral Red solution for 3 hours at 37° C. (Sigma Aldrich cat. no. N2889-100 mL). Neutral red was then aspirated from the wells and plaques were counted. The percent reduction in plaques was calculated relative to a no sera control. The reciprocal 50% neutralization titer was calculated by averaging the greatest dilution of mouse sera that had a greater than 50% reduction in plaques compared to a no sera control for each sera sample in a vaccination group.

### Statistical Analysis

Data was analyzed and presented using GraphPad Prism software. Statistical tests that were applied to each experiment are denoted in the appropriate figure legend. For all bar plots, unpaired Mann-Whitney tests were used. For survival plots, Mantel-Cox tests were used. All experiments were performed using a minimum of four samples, unless otherwise noted.

### Results:

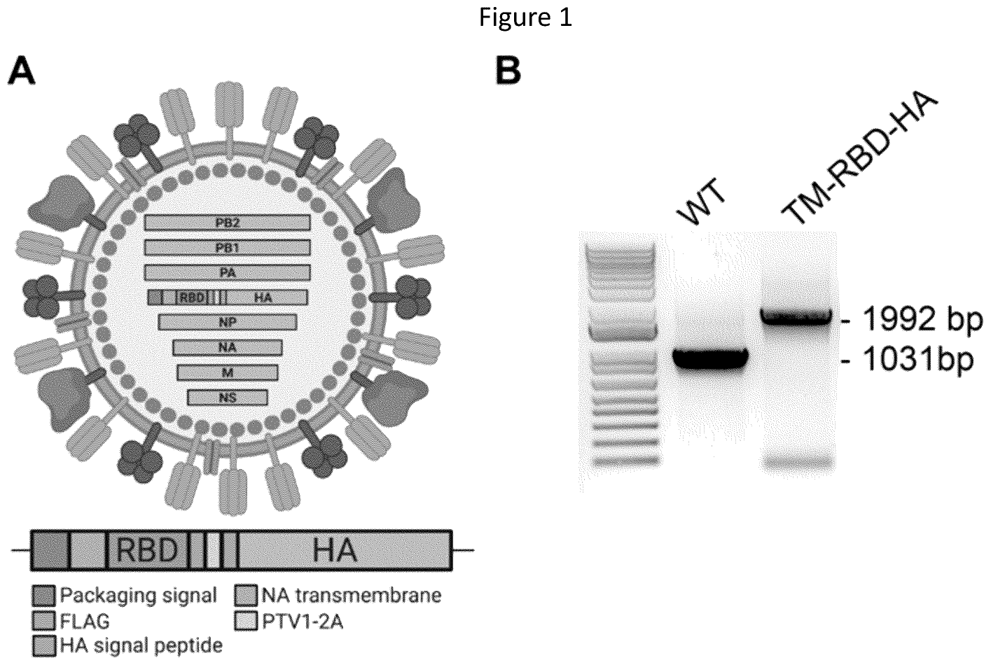

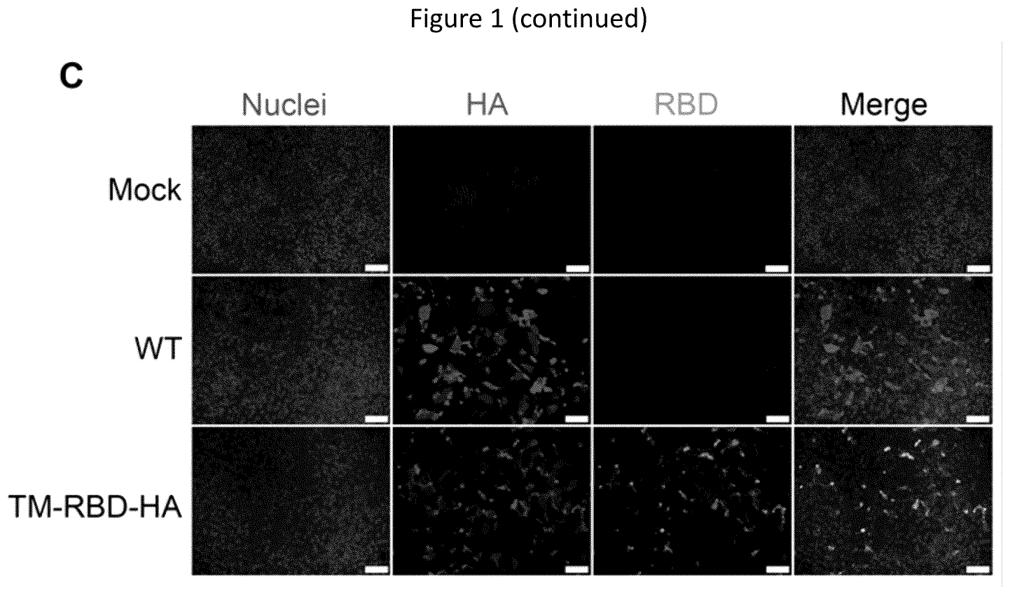

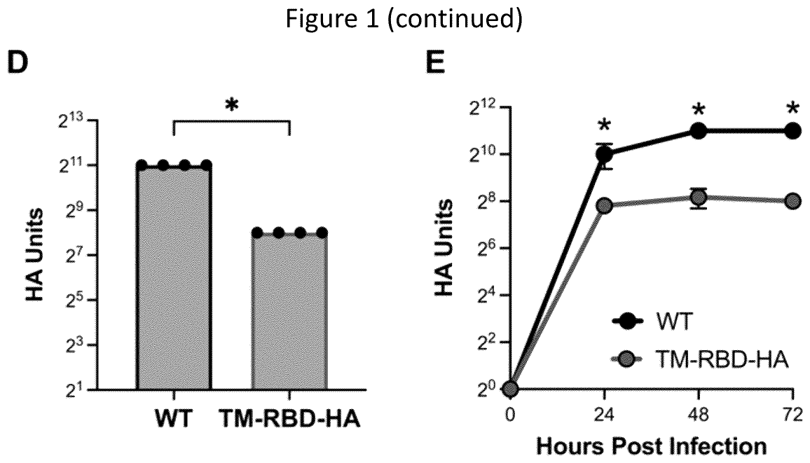

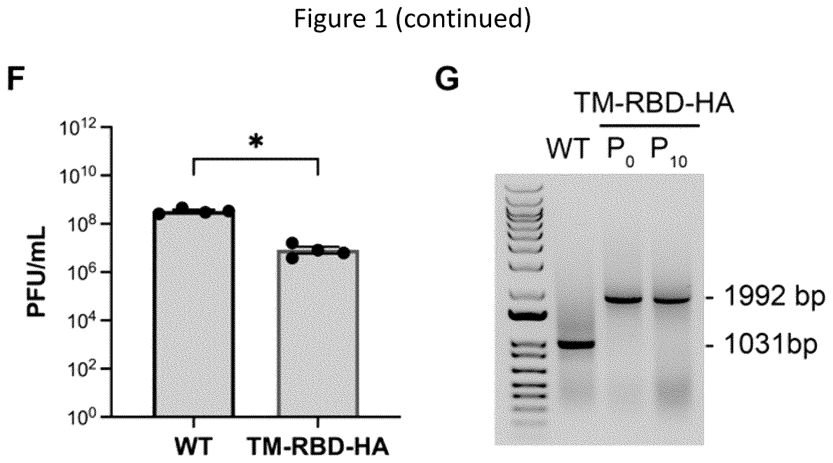

Generation of an H1N1 IAV encoding the SARS-CoV-2 RBD Our initial goal was to develop a strain of IAV that would encode a gene for the receptor binding domain (RBD) of the major antigen of SARS-CoV-2, the spike (S) glycoprotein^{26,27}. We wanted this gene to be expressed at high levels and for the translated SARS-CoV-2 antigen to be incorporated into an IAV particle such that the resulting vaccine could be administered as either a live attenuated or inactivated vaccine, formulations that are both currently approved for use in humans²⁸. We have previously published viral genetic approaches that allow for the incorporation of a foreign open reading frame into the viral genome²⁹⁻³¹. Using the mouse-adapted A/Puerto Rico/8/1934 (PR8) H₁N1 IAV, a strain that is considered to be incapable of causing disease in humans³² as the genetic background, we inserted the SARS-CoV-2 RBD (<18% of the full-length S protein) 5′ to the viral hemagglutinin (HA) ORF (FIG. 1A). This approach and genetic locus in IAV is associated with high expression of a foreign gene²⁹. To facilitate incorporation of the SARS-CoV-2 RBD onto nascent viral particles, we fused the ORF to the N-terminal sequence from the IAV neuraminidase (NA) protein which contains the transmembrane domain and cytoplasmic tail of that protein. A picornaviral 2A motif was included between the RBD and HA proteins to ensure that these proteins were co-translationally separated and independently packaged onto the viral particle (FIG. 1A).

We rescued the virus and verified appropriate insertion of the SARS-CoV-2 RBD ORF into the HA-encoding IAV segment via RT-PCR (FIG. 1B). To determine if the RBD was being expressed and localized appropriately to the plasma membrane, we infected cells, fixed them (but avoided permeabilization of the cell membrane), and then incubated them with antibodies against the IAV HA protein and the SARS-CoV-2 RBD. While infection with both non-modified wild-type (WT) IAV as well as the recombinant vaccine strain (TM-RBD-HA) led to expression of HA on the cell surface, membrane-anchored SARS-CoV-2 RBD was only detectable after infection with the TM-RBD-HA virus (FIG. 1C). In multicycle growth assays, the TM-RBD-HA virus efficiently amplified itself, although with delayed kinetics and a lower endpoint titer compared to WT PR8 (FIG. 1D-F). Finally, we wanted to ensure that the SARS-CoV-2 RBD ORF would remain stable in the viral genome. After 10 serial passages, the SARS-CoV-2 RBD ORF was amplified via RT-PCR with no apparent changes to its size (FIG. 1G). DNA sequencing of this RT-PCR product revealed no mutations in the SARS-CoV-2 RBD nor in the flanking genetic elements. Thus, we successfully developed an IAV vaccine that incorporates a membrane-anchored SARS-CoV-2 RBD antigenic domain.

The SARS-CoV-2 RBD is Packaged onto IAV Particles without Disruption of Native Viral Proteins

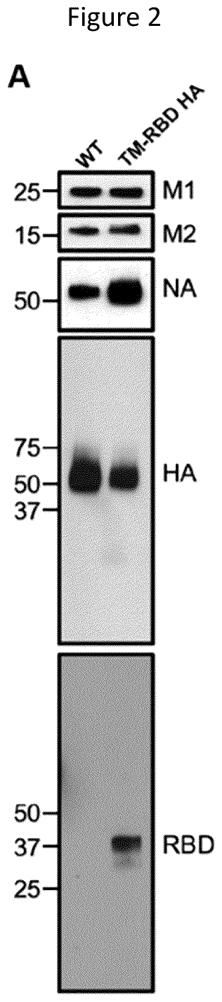

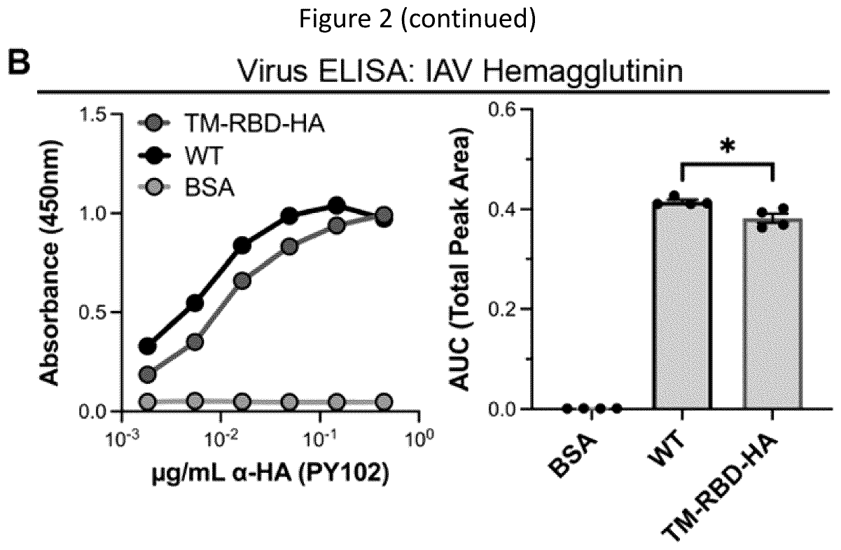

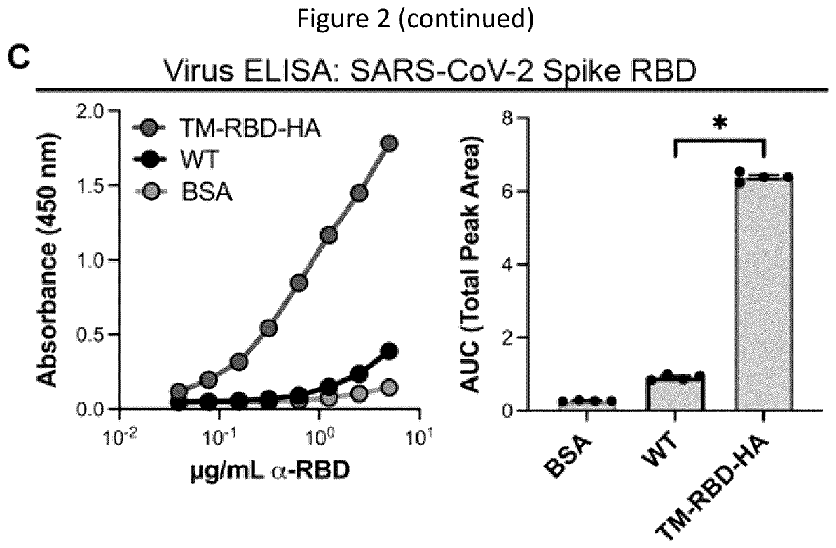

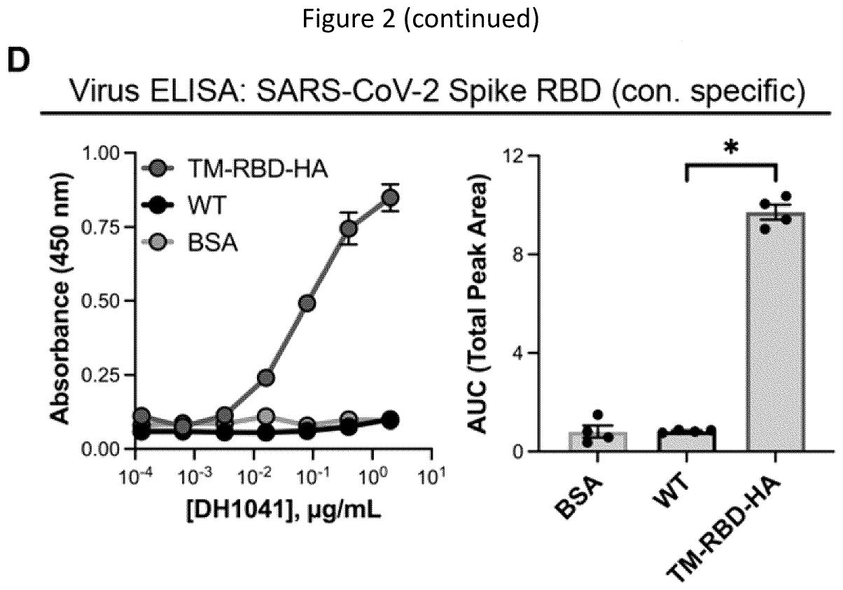

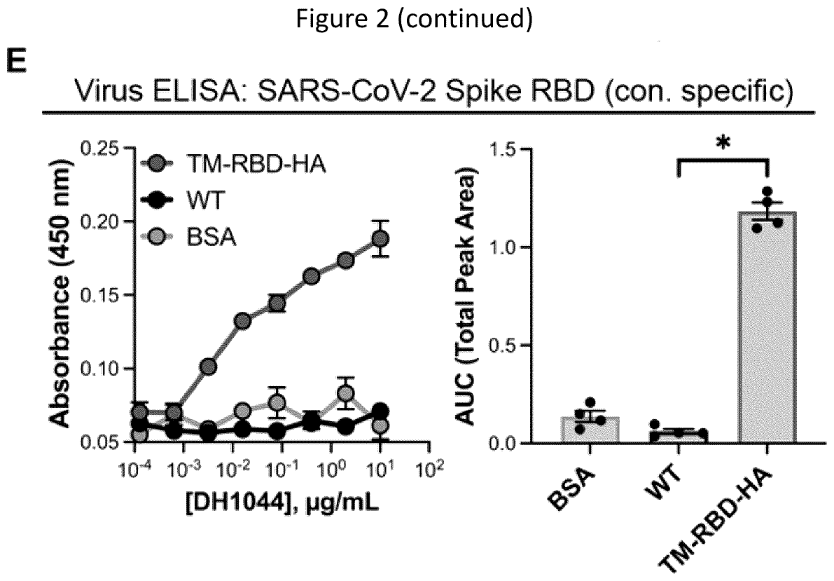

Next, we wanted to ensure that the membrane-anchored SARS-CoV-2 RBD was in fact incorporated into the viral particle. We therefore purified WT and TM-RBD-HA virus particles and analyzed their protein composition via western blot. We first normalized loading based on the levels of the IAV matrix (M1) protein in the two samples; the M1 protein forms the viral capsid and is not expected to be altered by changes to glycoprotein composition³³. As expected, we observed that SARS-CoV-2 RBD was detectable in the recombinant RBD virus, but not in the WT virus (FIG. 2A). To determine how incorporating another protein into the viral envelope influenced the native influenza viral surface proteins, we probed for expression of M2 as well as the HA and NA proteins. While the levels of the M2 protein were unchanged, there was a slight reduction in the amount of HA and an increase in the amount of NA packaged into nascent virions compared to WT virus (FIG. 2A). Thus, this genetic approach can facilitate packaging of a foreign protein onto a viral particle without dramatic effects on native viral protein incorporation.

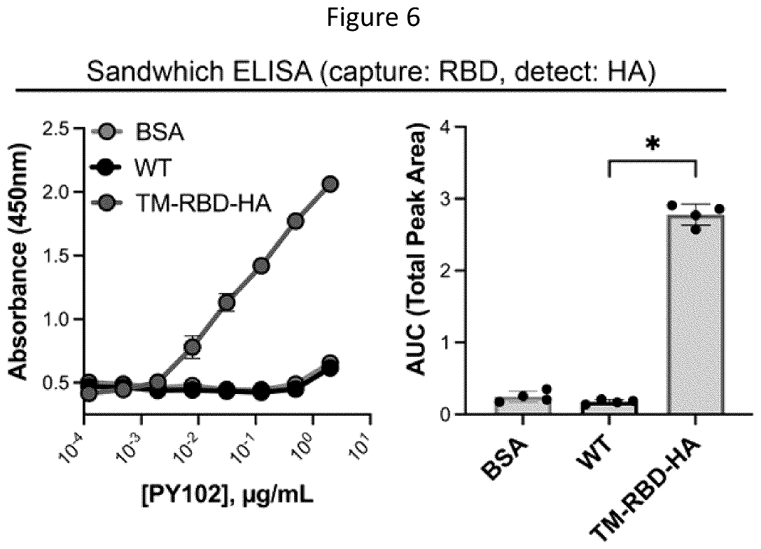

We next wanted to assess native glycoprotein virion packaging and the SARS-CoV-2 RBD secondary structure in the intact virions. By performing ELISA on purified virions, we observed a statistically significant but minor reduction of HA in the recombinant TM-RBD-HA virus compared to unmodified WT virus (FIG. 2B). We then probed for the SARS-CoV-2 RBD using antibodies that recognize either non-structural or structural epitopes. A non-conformation specific antibody raised against the RBD yielded strong signal only against the RBD virus (FIG. 2C). To verify that the TM-RBD fusion protein was folding correctly, we utilized DH1041 and DH1044, which are conformation specific human monoclonal antibodies that bind the SARS-CoV-2 RBD^{34,35}. Both of these antibodies specifically bound the TM-RBD-HA virus and not the parental WT IAV (FIG. 2D-E). To confirm that the RBD and HA proteins were packaged together into the same viral particles, we performed sandwich ELISAs. As expected, only the TM-RBD-HA samples had significant signal, indicating that virions expressing the RBD also expressed HA (FIG. 6). Taken together, these results indicate that the recombinant TM-RBD-HA virus packages properly folded SARS-CoV-2 RBD protein while maintaining the packaging of the other IAV envelope proteins HA, NA, M1, and M2.

The TM-RBD-HA Virus is Attenuated In Vitro In Vivo and does not Functionally Bind ACE2

Our IAV-based vaccine virus includes a small antigenic domain of the SARS-CoV-2 S protein that was not expected to confer any biologically relevant functionality. To ensure that this was the case, we first performed a multicycle growth curve analysis of WT A549 cells and A549 cells expressing the SARS-CoV-2 receptor protein, ACE2. In both experiments, the TM-RBD-HA virus had lower endpoint titers and reduced replication kinetics of similar magnitudes compared to WT virus (FIG. 3A). Next, we generated a panel of pseudotyped lentiviral vectors that express luciferase after successful cellular entry; these vectors allowed us to test the ability of the TM-RBD protein to mediate infection by itself. While both Vesicular stomatitis virus G protein (VSV-G) and the IAV HA and NA glycoproteins were sufficient to allow viral entry, the TM-RBD protein alone was insufficient to transduce either A549 or A549-ACE2 cells (FIG. 3B). In fact, the presence of TM-RBD with HA and NA significantly reduced the ability of the pseudotyped virus to transduce both cell lines, indicating that TM-RBD mediated alterations to native HA/NA glycoprotein packaging may potentially be a driver of the attenuation observed with our vaccine virus.

Next, to ensure that the entry of our vaccine virus was completely dependent on the canonical IAV HA-sialic acid interaction³⁶, we infected A549-ACE2 cells after treatment with Sialidase A, a technique which removes sialic acids from cell surface glycoproteins. For both the WT IAV and the TM-RBD-HA virus, treatment with Sialidase A decreased the infection rate by the same extent and to nearly the limit of detection (FIG. 3C, D). We also compared the neutralizing potency of PY102, an HA-targeting monoclonal antibody, against WT and TM-RBD-HA viruses using plaque reduction neutralization tests (PRNTs). The PRNT₅₀ of PY102 was unchanged when used against both the WT and TM-RBD-HA viruses (FIG. 3E). Finally, to ensure that our in vitro observations would be consistent with viral phenotypes in vivo, we infected K18-hACE2 mice that express hACE2 (i.e., the human SARS-CoV-2 receptor) and support SARS-CoV-2 infection³⁷ with multiple doses of both WT and TM-RBD-HA IAV. For all viral inoculums, murine morbidity and mortality was significantly reduced for the TM-RBD-HA virus compared to WT virus (FIG. 3F, G). Thus, the presence of the TM-RBD on the vaccine virus particle conferred no measurable benefit to the entry of the virus; in fact, attenuation and modest inhibition of entry specifically could be measured in our assays.

### Live-Attenuated TM-RBD-HA Vaccination Provides Improved Immunity Against IAV and SARS-CoV-2 Relative to Inactivated Vaccine Formulations

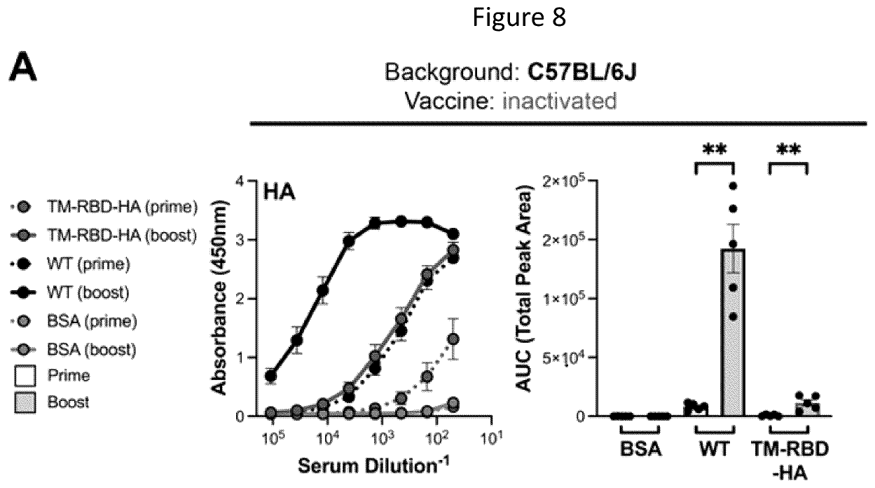

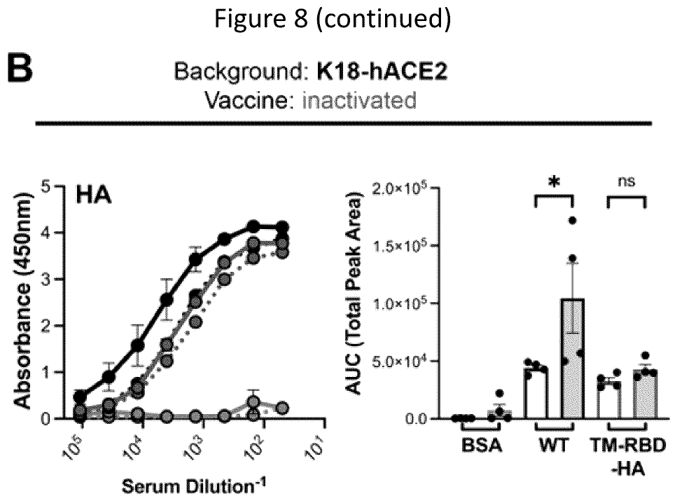

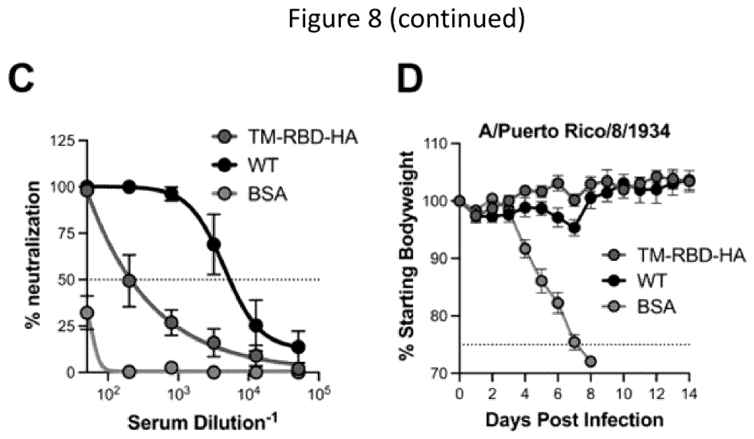

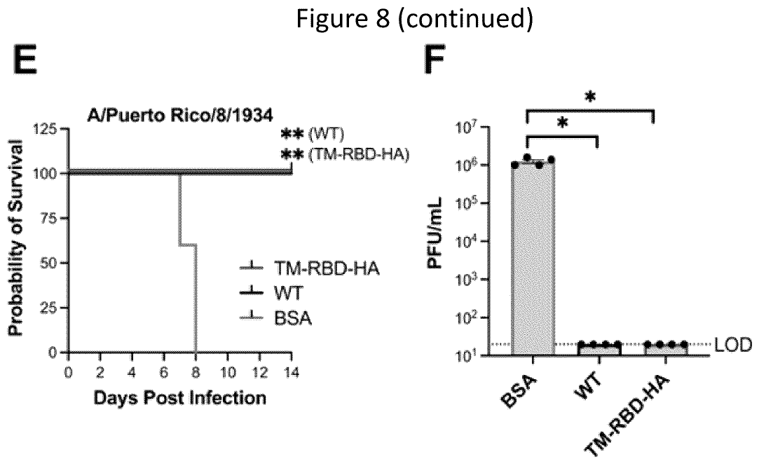

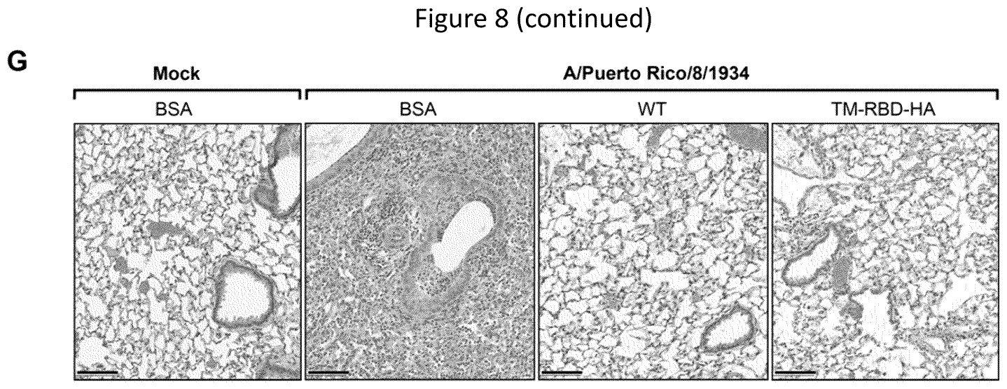

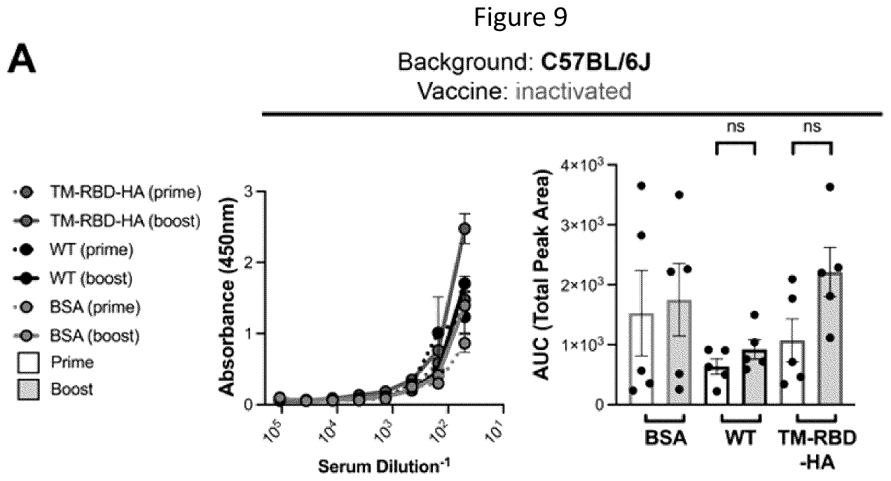

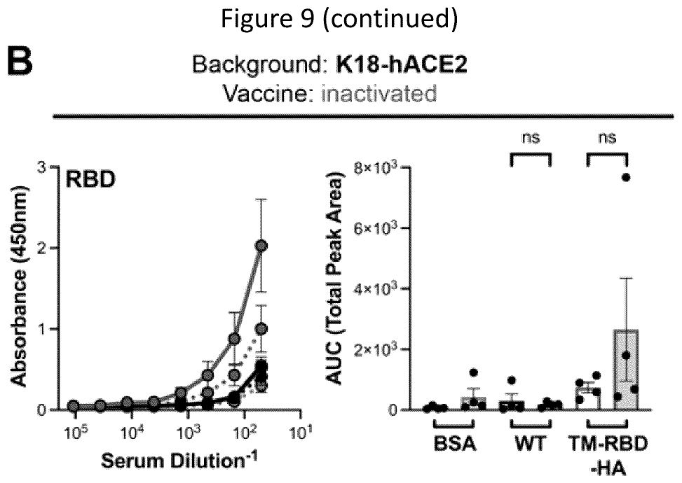

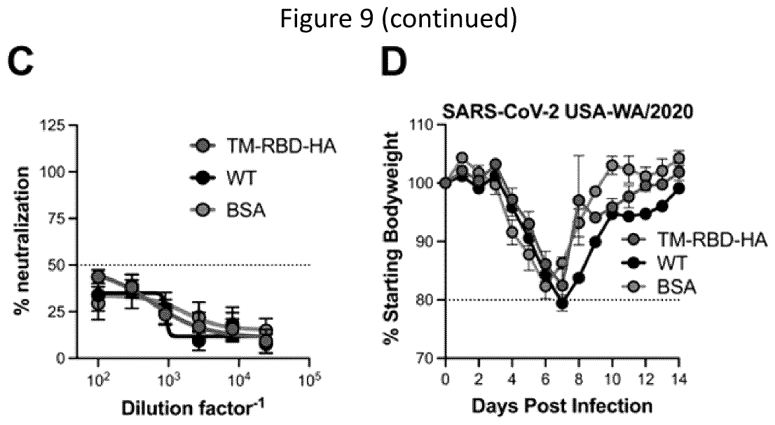

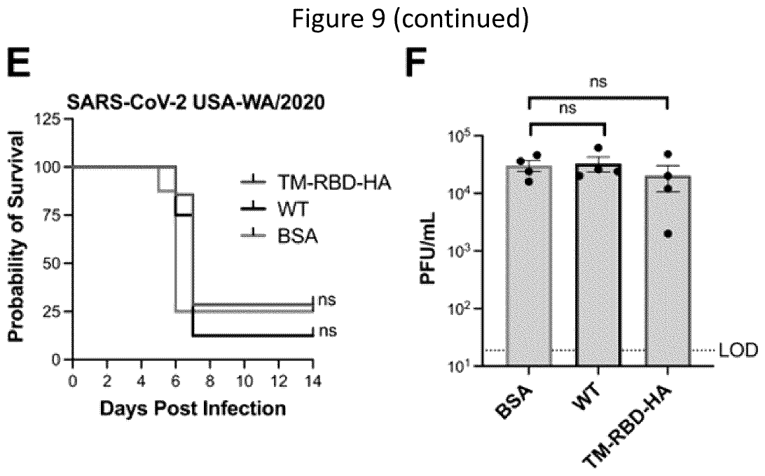

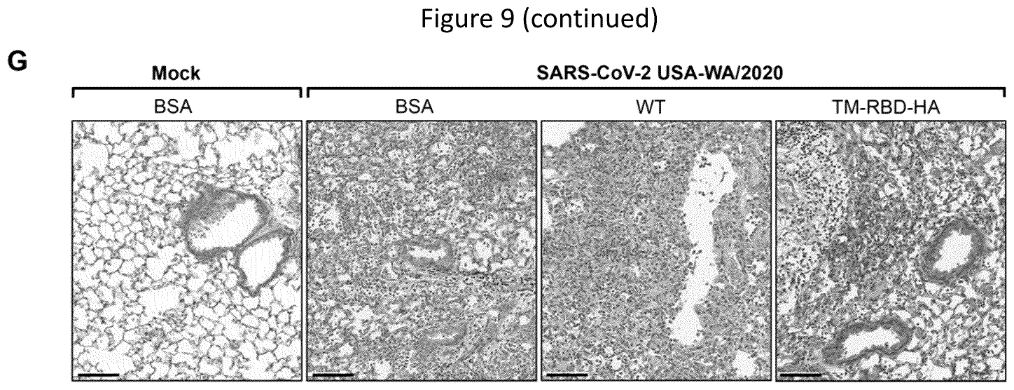

Our next step was to define the immune responses after vaccination with the TM-RBD-HA virus. First, we prepared inactivated whole-virion vaccines by treating virus with formalin. Although standard inactivated influenza vaccines are typically “split-inactivated” by detergent treatment, the immunogenicity of formalin-inactivated influenza vaccines has shown to induce similar responses³⁸. We first performed an inactivated prime-boost regimen followed by serum IgG characterization or challenge with a lethal dose of IAV or SARS-CoV-2 (FIG. 7). IAV-directed neutralizing serum antibodies as well as protection from IAV challenge were elicited by both vaccines (FIG. 8). SARS-CoV-2 RBD reactive serum IgG antibodies were detectable specifically in mice vaccinated with an inactivated preparation of the TM-RBD-HA virus, however the titers were insufficient to neutralize SARS-CoV-2 virus or protect from lethal challenge (FIG. 9).

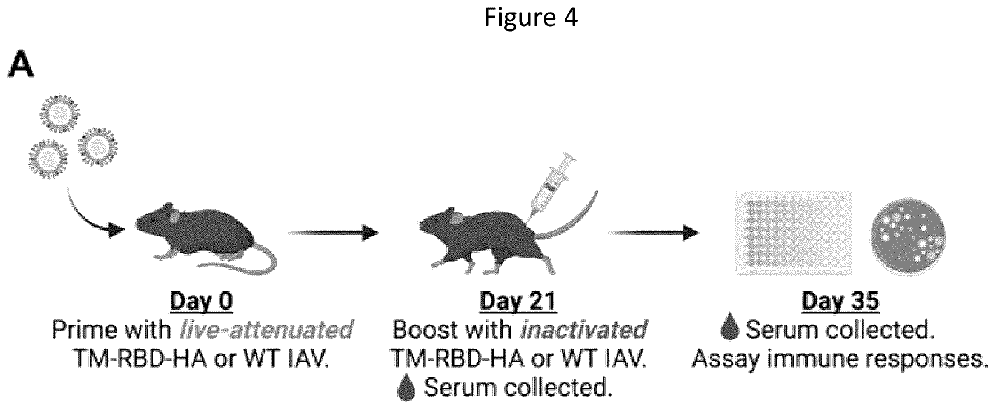

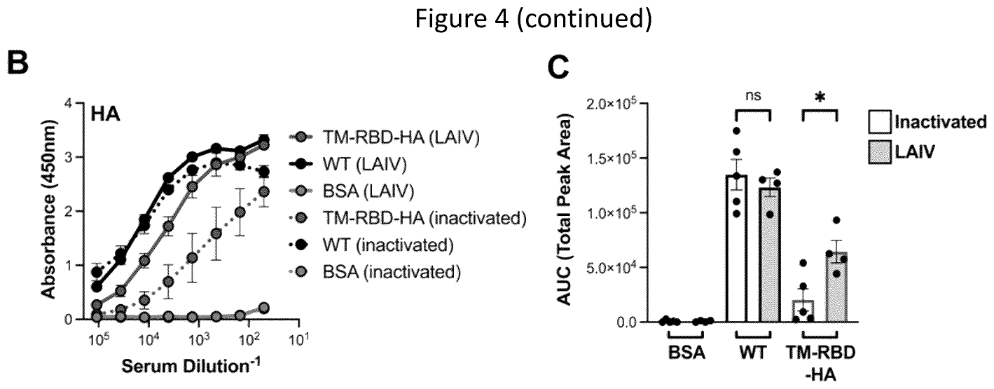

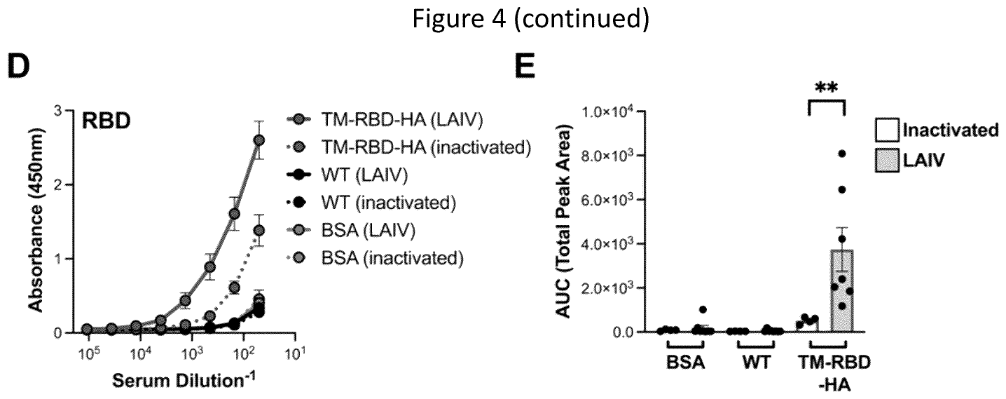

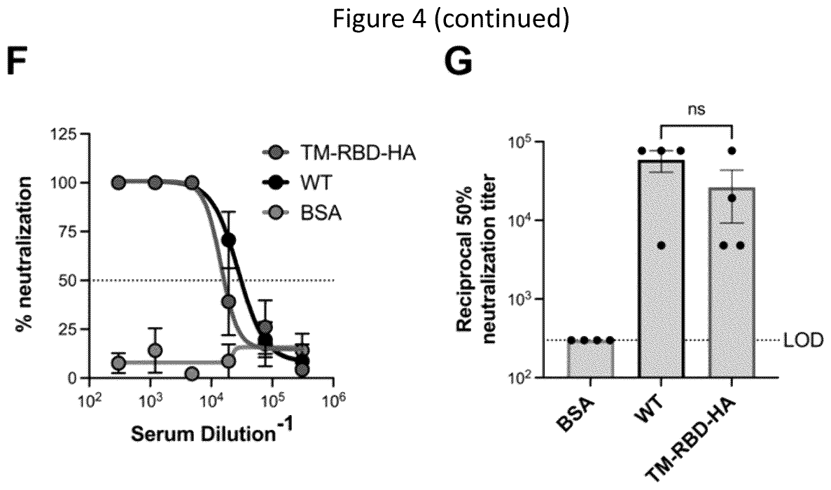

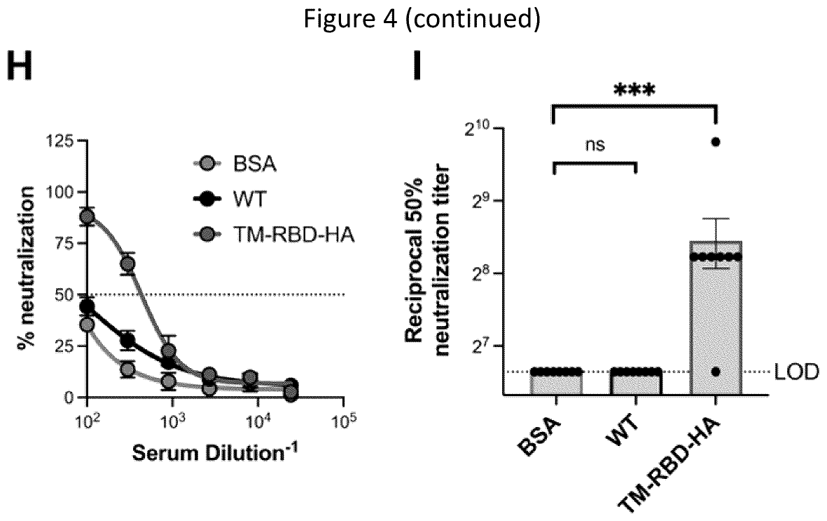

Live-attenuated influenza virus vaccination has been reported to provide superior protection relative to inactivated formulations in humans³⁹. Since our vaccine virus was naturally attenuated by addition of the RBD antigen but provided sub-optimal SARS-CoV-2 protection as an inactivated formulation, we altered our vaccination strategy. We first administered an intranasal live-attenuated influenza vaccine followed by an inactivated boost 21 days later (FIG. 4A). HA and RBD directed antibody responses elicited by this vaccination strategy were then measured. In all cases, while antigen reactive antibodies were detected after the prime, their levels were increased after inactivated virus boost (FIG. 10). Further, direct comparison of the inactivated and LAIV vaccination strategies demonstrated that indeed, the LAIV prime followed by inactivated boost led to higher magnitude of both IAV and RBD reactive antibodies (FIG. 4B-E). As with the inactivated formulation, the IAV reactive antibodies elicited by LAIV were also functional, and the sera from TM-RBD-HA vaccinated mice were able to neutralize authentic IAV similarly to sera from WT-vaccinated mice (FIG. 4F, G). In contrast to the inactivated vaccination approach however, PRNT analyses with SARS-CoV-2 showed that the post-vaccination sera were able to efficiently neutralize the virus (FIG. 4H, I).

Vaccination with Live TM-RBD-HA Virus Provides Protective Immunity Against IAV and SARS-CoV-2

After demonstrating the presence of serum neutralizing antibodies against both IAV and SARS-CoV-2, we next sought to determine if these responses would be sufficient to provide protection from lethal IAV and SARS-CoV-2 challenge. C57BL/6J or K18-hACE2 mice were, therefore, LAIV primed and boosted with inactivated virus or BSA (FIG. 5A). The vaccinated C57BL/6J mice were then challenged with a lethal dose of A/Puerto Rico/8/1934 IAV. In contrast to the BSA control vaccinated animals, animals vaccinated with either WT or TM-RBD-HA virus all survived the challenge and were protected from any detectable morbidity (FIG. 5B, C). Infectious IAV particles were also reduced to below the limit of detection in vaccinated mouse lung homogenates compared to more than 105 PFU/mL of lung homogenate in unvaccinated mice (FIG. 5D). And, as expected, histological analysis of lung tissue from mice that received either WT or TM-RBD-HA vaccines were indistinguishable from mock infected animals (FIG. 5E).

In parallel, the LAIV vaccinated K18-hACE2 mice were challenged with a lethal dose of SARS-CoV-2. Animals vaccinated with WT virus or BSA showed rapid weight loss and predominantly succumbed to the infection while the TM-RBD-HA mice displayed no measurable weight loss, and all animals survived the challenge (FIG. 5F, G). High levels of infectious SARS-CoV-2 were detectable in lung homogenates of WT-vaccinated animals, while the burden in TM-RBD-HA-vaccinated animals was significantly reduced (FIG. 5H). Lung tissue morphology was also investigated after challenge with SARS-CoV-2 and mice that received WT vaccine harbored severe lung infiltration/inflammation compared to mice that received TM-RBD-HA vaccine (FIG. 5I). Taken together, these data demonstrate that a LAIV vaccination with our combination TM-RBD-HA yields strong protective immunity against both IAV and SARS-CoV-2.

### Discussion

In general, the development and production of novel vaccines is labor intensive, costly, and difficult to scale upon high demand. There are many different influenza vaccines used worldwide, including split-inactivated, live-attenuated, and recombinant protein-based vaccines. The existing infrastructure for producing influenza vaccines is highly optimized and capable of delivering more than a billion doses per year⁴⁰. To demonstrate the feasibility of leveraging the influenza vaccine infrastructure for the production of other vaccines, we have shown that a laboratory adapted IAV vaccine backbone (A/Puerto Rico/8/1934) can be used to express and package the receptor binding domain from the spike protein of SARS-CoV-2. Vaccination of mice with this vaccine administered as a LAIV followed by an inactivated boost elicited neutralizing antibodies and protected against lethal challenge from both IAV and SARS-CoV-2. Thus, an influenza virus-based vaccine platform may be a practical solution, allowing a combination seasonal vaccine to be produced in an analogous manner to standard influenza vaccines.

Our data are in general agreement with previous work that has shown that influenza viruses can be used as a vector to express other antigens, including additional influenza proteins^{29,41,42} and antigens and/or epitopes from pathogens as divergent as Mycobacterium tuberculosis⁴³ and Chlamydia trachomatis⁴⁴. Such studies demonstrate that IAV can be modified to elicit non-influenza directed responses from the immune system. Packaging of the SARS-CoV-2 RBD gene into an influenza particle has also been previously described. Loes et al. successfully generated a live-attenuated ΔNA(RBD) virus that packages the SARS-CoV-2 RBD but lacks NA and requires a mutation in the HA coding sequence to reduce its affinity for sialic acid⁴⁵. Vaccination with this virus was able to induce neutralizing SARS-CoV-2 antibodies. Our approach has several advantages over the approach taken in this earlier study, including the ability to elicit NA-directed immunity and the ability to package the non-influenza antigen onto viral particles rather than restricting its expression to infected cells. But, together, our work and the work presented by Loes et al. clearly demonstrate that influenza virus-based vaccines can elicit protection against other pathogens.

An outstanding question is whether boosting the RBD-directed immune response will be sufficient to mediate protection against different SARS-CoV-2 variants. If so, an added benefit to including the RBD in the influenza vaccine is that RBDs from several different variants could be included. Most current influenza vaccines contain four influenza strains (i.e., two IAV and two IBV strains), allowing up to four different SARS-CoV-2 variant RBDs to be included. Finally, the exact mechanisms of protection by our vaccine remain incompletely defined. LAIVs can elicit both strong antibody and cytotoxic T-cell responses. Although cell-mediated immune responses were not specifically measured in this study, these activities likely contributed to protection. The magnitude and nature of such responses will need to be examined further.

In conclusion, influenza-based, multi-valent vaccines represent a generalizable approach to reduce the time and manufacturing requirements for developing novel vaccines. Since the current influenza vaccine is composed of three or four distinct strains⁵, this approach could be further multiplexed to elicit responses against more than two pathogens or multiple strains of the same pathogens. While there remain questions to be answered and technical challenges to overcome, influenza virus-based vaccines may be an attractive approach to produce and package antigens that are difficult to purify or are poorly immunogenic on their own. Continued work on this and other generalizable vaccine platforms will not only help with the current response to the COVID-19 pandemic but will help poise us for rapid response to future epidemic/pandemic outbreaks.

### REFERENCES

1 Harding, A. T. & Heaton, N. S. Efforts to Improve the Seasonal Influenza Vaccine. Vaccines (Basel) 6, doi:10.3390/vaccines6020019 (2018).
2 Trombetta, C. M., Gianchecchi, E. & Montomoli, E. Influenza vaccines: Evaluation of the safety profile. Hum Vaccin Immunother 14, 657-670, doi:10.1080/21645515.2017.1423153 (2018).
3 Savidan, E., Chevat, C. & Marsh, G. Economic evidence of influenza vaccination in children. Health Policy 86, 142-152, doi:10.1016/j.healthpol.2007.09.009 (2008).
4 Sullivan, S. G., Price, O. H. & Regan, A. K. Burden, effectiveness and safety of influenza vaccines in elderly, paediatric and pregnant populations. Ther Adv Vaccines Immunother 7, 2515135519826481, doi:10.1177/2515135519826481 (2019).
5 Wei, C. J. et al. Next-generation influenza vaccines: opportunities and challenges. Nat Rev Drug Discov 19, 239-252, doi:10.1038/s41573-019-0056-x (2020).
6 Sridhar, S., Brokstad, K. A. & Cox, R. J. Influenza Vaccination Strategies: Comparing Inactivated and Live Attenuated Influenza Vaccines. Vaccines (Basel) 3, 373-389, doi:10.3390/vaccines3020373 (2015).
7 Hoft, D. F. et al. Live and inactivated influenza vaccines induce similar humoral responses, but only live vaccines induce diverse T-cell responses in young children. J Infect Dis 204, 845-853, doi:10.1093/infdis/jir436 (2011).
8 Zens, K. D., Chen, J. K. & Farber, D. L. Vaccine-generated lung tissue-resident memory T cells provide heterosubtypic protection to influenza infection. JCI Insight 1, doi:10.1172/jci.insight.85832 (2016).
9 Sasaki, S. et al. Comparison of the influenza virus-specific effector and memory B-cell responses to immunization of children and adults with live attenuated or inactivated influenza virus vaccines. J Virol 81, 215-228, doi:10.1128/JVI.01957-06 (2007).
10 Nogales, A. & Martinez-Sobrido, L. Reverse Genetics Approaches for the Development of Influenza Vaccines. Int J Mol Sci 18, doi:10.3390/ijms18010020 (2016).
11 Fiege, J. K. & Langlois, R. A. Investigating influenza A virus infection: tools to track infection and limit tropism. J Virol 89, 6167-6170, doi:10.1128/JVI.00462-15 (2015).
12 Dumm, R. E. & Heaton, N. S. The Development and Use of Reporter Influenza B Viruses. Viruses-Basel 11, doi:ARTN 73610.3390/v11080736 (2019).
13 Jenkins, M. R., Webby, R., Doherty, P. C. & Turner, S. J. Addition of a prominent epitope affects influenza a virus-specific CD8(+) T cell immunodominance hierarchies when antigen is limiting. Journal of Immunology 177, 2917-2925, doi:DOI 10.4049/jimmunol.177.5.2917 (2006).
14 Hu, B., Guo, H., Zhou, P. & Shi, Z. L. Characteristics of SARS-CoV-2 and COVID-19. Nat Rev Microbiol 19, 141-154, doi:10.1038/s41579-020-00459-7 (2021).
15 Dong, Y. et al. A systematic review of SARS-CoV-2 vaccine candidates. Signal Transduct Target Ther 5, 237, doi:10.1038/s41392-020-00352-y (2020).
16 Sadoff, J. et al. Interim Results of a Phase 1-2a Trial of Ad26.COV2.S Covid-19 Vaccine. N Engl J Med, doi:10.1056/NEJMoa2034201 (2021).
17 Baden, L. R. et al. Efficacy and Safety of the mRNA-1273 SARS-CoV-2 Vaccine. N Engl J Med 384, 403-416, doi:10.1056/NEJMoa2035389 (2021).
18 Polack, F. P. et al. Safety and Efficacy of the BNT162b2 mRNA Covid-19 Vaccine. N Engl J Med 383, 2603-2615, doi:10.1056/NEJMoa2034577 (2020).
19 Rubin, R. COVID-19 Vaccines vs Variants-Determining How Much Immunity Is Enough. Jama-J Am MedAssoc, doi:10.1001/jama.2021.3370 (2021).
20 Mlcochova, P. et al. SARS-CoV-2 B.1.617.2 Delta variant replication and immune evasion. Nature 599, 114-119, doi:10.1038/s41586-021-03944-y (2021).
21 Wang, Y. et al. The significant immune escape of pseudotyped SARS-CoV-2 variant Omicron. Emerg Microbes Infect 11, 1-5, doi:10.1080/22221751.2021.2017757 (2022).
22 Honda-Okubo, Y. et al. Severe acute respiratory syndrome-associated coronavirus vaccines formulated with delta inulin adjuvants provide enhanced protection while ameliorating lung eosinophilic immunopathology. J Virol 89, 2995-3007, doi:10.1128/JVI.02980-14 (2015).
23 Poland, G. A., Ovsyannikova, I. G. & Kennedy, R. B. SARS-CoV-2 immunity: review and applications to phase 3 vaccine candidates. Lancet 396, 1595-1606, doi:10.1016/50140-6736(20)32137-1 (2020).
24 Sariol, A. & Perlman, S. Lessons for COVID-19 Immunity from Other Coronavirus Infections. Immunity 53, 248-263, doi:10.1016/j.immuni.2020.07.005 (2020).
25 Huang, A. T. et al. A systematic review of antibody mediated immunity to coronaviruses: kinetics, correlates of protection, and association with severity. Nat Commun 11, 4704, doi:10.1038/s41467-020-18450-4 (2020).
26 Lan, J. et al. Structure of the SARS-CoV-2 spike receptor-binding domain bound to the ACE2 receptor. Nature 581, 215-+, doi:10.1038/s41586-020-2180-5 (2020).
27 Tai, W. B. et al. Characterization of the receptor-binding domain (RBD) of 2019 novel coronavirus: implication for development of RBD protein as a viral attachment inhibitor and vaccine. Cel/Mol Immunol 17, 613-620, doi:10.1038/s41423-020-0400-4 (2020).
28 Soema, P. C., Kompier, R., Amorij, J. P. & Kersten, G. F. Current and next generation influenza vaccines: Formulation and production strategies. Eur J Pharm Biopharm 94, 251-263, doi:10.1016/j.ejpb.2015.05.023 (2015).
29 Harding, A. T., Heaton, B. E., Dumm, R. E. & Heaton, N. S. Rationally Designed Influenza Virus Vaccines That Are Antigenically Stable during Growth in Eggs. Mbio 8, doi:ARTN e00669-1710.1128/mBio.00669-17 (2017).
30 Heaton, N. S. et al. In vivo bioluminescent imaging of influenza a virus infection and characterization of novel cross-protective monoclonal antibodies. J Virol 87, 8272-8281, doi:10.1128/JVI.00969-13 (2013).
31 Dumm, R. E. et al. Non-lytic clearance of influenza B virus from infected cells preserves epithelial barrier function. Nat Commun 10, 779, doi:10.1038/s41467-019-08617-z (2019).
32 Beare, A. S., Schild, G. C. & Craig, J. W. Trials in Man with Live Recombinants Made from a-Pr-8-34 (H0 N1) and Wild H-3 N2 Influenza-Viruses. Lancet 2, 729-732 (1975).
33 Selzer, L., Su, Z. M., Pintilie, G. D., Chiu, W. & Kirkegaard, K. Full-length three-dimensional structure of the influenza A virus M1 protein and its organization into a matrix layer. PlosBiol 18, doi:ARTN e300082710.1371/journal.pbio.3000827 (2020).
34 Li, D. et al. In vitro and in vivo functions of SARS-CoV-2 infection-enhancing and neutralizing antibodies. Cell 184, 4203-4219 e4232, doi:10.1016/j.cell.2021.06.021 (2021).
35 Saunders, K. O. et al. Neutralizing antibody vaccine for pandemic and pre-emergent coronaviruses. Nature 594, 553-559, doi:10.1038/s41586-021-03594-0 (2021).
36 Weis, W. et al. Structure of the influenza virus haemagglutinin complexed with its receptor, sialic acid. Nature 333, 426-431, doi:10.1038/333426a0 (1988).
37 Winkler, E. S. et al. SARS-CoV-2 infection of human ACE2-transgenic mice causes severe lung inflammation and impaired function (vol 51, μg 831, 2020). Nat Immunol 21, 1470-1470, doi:10.1038/s41590-020-0794-2 (2020).
38 Okamoto, S. et al. Intranasal immunization with a formalin-inactivated human influenza A virus whole-virion vaccine alone and intranasal immunization with a split-virion vaccine with mucosal adjuvants show similar levels of cross-protection. Clin Vaccine Immunol 19, 979-990, doi:10.1128/CVI.00016-12 (2012).
39 Belshe, R. B. et al. Live attenuated versus inactivated influenza vaccine in infants and young children. N Engl J Med 356, 685-696, doi:10.1056/NEJMoa065368 (2007).
40 Sparrow, E. et al. Global production capacity of seasonal and pandemic influenza vaccines in 2019. Vaccine 39, 512-520, doi:10.1016/j.vaccine.2020.12.018 (2021).
41 Pena, L. et al. Influenza Viruses with Rearranged Genomes as Live-Attenuated Vaccines. Journal of Virology 87, 5118-5127, doi:10.1128/Jvi.02490-12 (2013).
42 Gao, Q., Lowen, A. C., Wang, T. T. & Palese, P. A nine-segment influenza a virus carrying subtype H₁ and H₃ hemagglutinins. J Virol 84, 8062-8071, doi:10.1128/JVI.00722-10 (2010).
43 Sereinig, S. et al. Influenza virus NS vectors expressing the Mycobacterium tuberculosis ESAT-6 protein induce CD4+Th1 immune response and protect animals against tuberculosis challenge. Clin Vaccine Immunol 13, 898-904, doi:10.1128/CVI.00056-06 (2006).
44 He, Q. et al. Live-attenuated influenza viruses as delivery vectors for Chlamydia vaccines. Immunology 122, 28-37, doi:10.1111/j.1365-2567.2007.02608.x (2007).
45 Loes, A. N., Gentles, L. E., Greaney, A. J., Crawford, K. H. D. & Bloom, J. D. Attenuated Influenza Virions Expressing the SARS-CoV-2 Receptor-Binding Domain Induce Neutralizing Antibodies in Mice. Viruses 12, doi:10.3390/v12090987 (2020).
46 Sun, W. et al. A Newcastle disease virus expressing a stabilized spike protein of SARS-CoV-2 induces protective immune responses. Nat Commun 12, 6197, doi:10.1038/s41467-021-26499-y (2021).
47 Mendonca, S. A., Lorincz, R., Boucher, P. & Curiel, D. T. Adenoviral vector vaccine platforms in the SARS-CoV-2 pandemic. Npj Vaccines 6, doi:ARTN 9710.1038/s41541-021-00356-x (2021).
48 Frantz, P. N. et al. A live measles-vectored COVID-19 vaccine induces strong immunity and protection from SARS-CoV-2 challenge in mice and hamsters. Nature Communications 12, doi:ARTN 627710.1038/s41467-021-26506-2 (2021).
49 Lu, M. J. et al. A safe and highly efficacious measles virus-based vaccine expressing SARS-CoV-2 stabilized prefusion spike. P Natl Acad Sci USA 118, doi:ARTN e202615311810.1073/pnas.2026153118 (2021).

1. An engineered polynucleotide comprising:
a) a first polynucleotide encoding an influenza neuraminidase (NA) protein comprising a cytoplasmic tail and a transmembrane domain fused to a receptor binding domain (RBD) of a SARS-CoV-2 spike protein; and
b) a second polynucleotide encoding an influenza hemagglutinin (HA) protein;
wherein the first polynucleotide is linked to the second polynucleotide by a third polynucleotide encoding a linker peptide.

2. (canceled)

3. The engineered polynucleotide of claim 1, wherein the engineered polynucleotide is part of segment 4 from an influenza virus.

4. The engineered polynucleotide of claim 1, further comprising an influenza virus packaging signal.

5. (canceled)

6. The engineered polynucleotide of claim 4, wherein the engineered polynucleotide comprises from 5′ to 3′ on the sense strand:
a) a viral 5′ untranslated region (UTR);
b) the influenza virus packaging signal;
c) the first polynucleotide;
d) the third polynucleotide;
e) the second polynucleotide; and
f) a viral 3′ UTR.

7. The engineered polynucleotide of claim 1, wherein
a) the portion of the influenza NA protein comprises amino acids 1-40 of an influenza NA protein;
b) the RBD comprises about 150-250 amino acid residues of the SARS-CoV-2 spike protein;
c) the linker peptide comprises a protein tag or a self-cleaving polypeptide;
d) the influenza HA protein is an HA subtype 1 (HA1) protein or an HA subtype 3 (HA3) protein: or
e) any combination of (a)-(d).

8. The engineered polynucleotide of claim 7, wherein
a) the portion of the influenza NA protein comprises SEQ ID NO: 10;
b) the RBD comprises SEQ ID NO: 11;
c) the linker peptide comprises SEQ ID NO: 12 or SEQ ID NO: 13;
d) the influenza HA protein comprises SEQ ID NO: 14; or
e) any combination of (a)-(d).

9. (canceled)

10. (canceled)

11. (canceled)

12. (canceled)

13. (canceled)

14. (canceled)

15. (canceled)

16. (canceled)

17. The engineered polynucleotide of claim 1, wherein the engineered polynucleotide comprises SEQ ID NO: 1 or a sequence having at least 60% sequence identity to SEQ ID NO: 1.

18. A plasmid comprising the engineered polynucleotide of claim 1.

19. A plasmid composition comprising plasmids encoding influenza virus segments 1, 2, 3, 5, 6, 7, and 8 and the plasmid of claim 18.

20. A cell comprising the composition of claim 19.

21. An influenza virus produced by the cell of claim 20.

22. An influenza virus comprising the engineered polynucleotide of claim 1.

23. The influenza virus of claim 22, wherein the influenza virus expresses both the RBD of the SARS-CoV-2 spike protein and the influenza HA protein on its surface.

24. The influenza virus of claim 22, wherein the influenza virus can propagate itself in embryonated chicken eggs or in cell culture.

25. (canceled)

26. The influenza virus of claim 22, wherein the influenza virus is inactivated or attenuated.

27. A pharmaceutical composition comprising the influenza virus of claim 22 and a pharmaceutically acceptable carrier.

28. A method for generating an immune response to both influenza and SARS-CoV-2 in a subject, the method comprising administering a therapeutically effective amount of the influenza virus of claim 22 to the subject.

29. The method of claim 28, wherein the influenza virus is administered in a prime-boost vaccination scheme.

30. The method of claim 29, wherein the prime comprises live-attenuated influenza virus and the boost comprises inactivated influenza virus.

31. A method for producing an influenza virus, the method comprising:
a) transfecting the plasmid composition of claim 19 into a cell;
b) incubating the transfected cell; and
c) harvesting the influenza virus;
wherein the influenza virus expresses both the RBD of the SARS-CoV-2 spike protein and the influenza HA protein on its surface.


SEQUENCE LISTING (23 sequences)

SEQ ID NO: 1 - DNA, 2736 bp, Artificial Sequence
  Synthetic - engineered polypeptide derived from influenza A virus segment 4
agcaaaagcaggggaaaataaaaacaaccaaattgaaggcaaacctactggtcctgttaagtgcacttgcagctgcagttgcagacacaatttgtataggccaccatgaaccccaatcaaaaaattacaaccatcgggtctatctgcctcgtagtcggactgatatccctaattcttcagataggtaatatcatatctatctggatcagtcatagtatacagactagagtgcagccaacagagtcaatcgtgagattcccaaatatcacaaatctgtgcccattcggagaagtgttcaatgcaacaagattcgcatcagtgtacgcatggaacagaaagagaatctccaattgcgtggcagactactcagtgctgtacaattcagcatcattctcaacattcaaatgctacggagtgtcaccaacaaaacttaatgatctttgcttcacaaatgtgtatgcagattcattcgtgataagaggagatgaagtgagacaaatagcaccaggacaaacaggaaaaatagcagattataattataaacttccagatgatttcacagggtgcgtgatcgcatggaattcaaacaatctggactcaaaagtgggagggaattacaactacctgtatagactgttcagaaaatcaaatctcaaaccattcgaaagagacatctcaacagaaatctaccaagcaggatcaacaccatgcaatggagtggaaggattcaattgctatttcccactgcagtcatatggattccagccaacaaatggagtgggatatcaaccatatagagtggtggtgctttcattcgaacttcttcatgcaccagcaacagtgtgcggaccaaaaaaatcaacaaatcttgtgaaaaataaatgcgtgaatttcggcagtggggactataaggacgatgatgacaaaggatctggggctaccaacttcagtctcctcaaacaggccggagacgtggaagaaaatcctgggcctatgaaagcgaatttgttagttttactgtccgcgttggcggccgcggacgcagacacaatatgtataggctaccatgcgaacaattcaaccgacactgttgacacagtactcgagaagaatgtgacagtgacacactctgttaacctgctcgaagacagccacaacggaaaactatgtagattaaaaggaatagccccactacaattggggaaatgtaacatcgccggatggctcttgggaaacccagaatgcgacccactgcttccagtgagatcatggtcctacattgtagaaacaccaaactctgagaatggaatatgttatccaggagatttcatcgactatgaggagctgagggagcaattgagctcagtgtcatcattcgaaagattcgaaatatttcccaaagaaagctcatggcccaaccacaacacaaacggagtaacggcagcatgctcccatgaggggaaaagcagtttttacagaaatttgctatggctgacggagaaggagggctcatacccaaagctgaaaaattcttatgtgaacaaaaaagggaaagaagtccttgtactgtggggtattcatcacccgcctaacagtaaggaacaacagaatctctatcagaatgaaaatgcttatgtctctgtagtgacttcaaattataacaggagatttaccccggaaatagcagaaagacccaaagtaagagatcaagctgggaggatgaactattactggaccttgctaaaacccggagacacaataatatttgaggcaaatggaaatctaatagcaccaatgtatgctttcgcactgagtagaggctttgggtccggcatcatcacctcaaacgcatcaatgcatgagtgtaacacgaagtgtcaaacacccctgggagctataaacagcagtctcccttaccagaatatacacccagtcacaataggagagtgcccaaaatacgtcaggagtgccaaattgaggatggttacaggactaaggaacactccgtccattcaatccagaggtctatttggagccattgccggttttattgaagggggatggactggaatgatagatggatggtatggttatcatcatcagaatgaacagggatcaggctatgcagcggatcaaaaaagcacacaaaatgccattaacgggattacaaacaaggtgaacactgttatcgagaaaatgaacattcaattcacagctgtgggtaaagaattcaacaaattagaaaaaaggatggaaaatttaaataaaaaagttgatgatggatttctggacatttggacatataatgcagaattgttagttctactggaaaatgaaaggactctggatttccatgactcaaatgtgaagaatctgtatgagaaagtaaaaagccaattaaagaataatgccaaagaaatcggaaatggatgttttgagttctaccacaagtgtgacaatgaatgcatggaaagtgtaagaaatgggacttatgattatcccaaatattcagaagagtcaaagttgaacagggaaaaggtagatggagtgaaattggaatcaatggggatctatcagattctggcgatctactcaactgtcgccagttcactggtgcttttggtctccctgggggcaatcagtttctggatgtgttctaatggatctttgcagtgcagaatatgcatctgagattagaatttcagaaatatgaggaaaaacacccttgtttctact

SEQ ID NO: 2 - DNA, 32 bp, Artificial Sequence
  Synthetic - viral 5′ UTR
agcaaaagcaggggaaaataaaaacaaccaaa

SEQ ID NO: 3 - DNA, 67 bp, Artificial Sequence
  Synthetic - viral packaging signal
ttgaaggcaaacctactggtcctgttaagtgcacttgcagctgcagttgcagacacaatttgtatag

SEQ ID NO: 4 - DNA, 120 bp, Artificial Sequence
  Synthetic - portion of a neuraminidase (NA) protein comprising the transmembrane domain and cytoplasmic tail
atgaaccccaatcaaaaaattacaaccatcgggtctatctgcctcgtagtcggactgatatccctaattcttcagataggtaatatcatatctatctggatcagtcatagtatacagact

SEQ ID NO: 5 - DNA, 669 bp, Artificial Sequence
  Synthetic - receptor binding domain (RBD) of the spike protein from the SARS-CoV-2 Wuhan variant
agagtgcagccaacagagtcaatcgtgagattcccaaatatcacaaatctgtgcccattcggagaagtgttcaatgcaacaagattcgcatcagtgtacgcatggaacagaaagagaatctccaattgcgtggcagactactcagtgctgtacaattcagcatcattctcaacattcaaatgctacggagtgtcaccaacaaaacttaatgatctttgcttcacaaatgtgtatgcagattcattcgtgataagaggagatgaagtgagacaaatagcaccaggacaaacaggaaaaatagcagattataattataaacttccagatgatttcacagggtgcgtgatcgcatggaattcaaacaatctggactcaaaagtgggagggaattacaactacctgtatagactgttcagaaaatcaaatctcaaaccattcgaaagagacatctcaacagaaatctaccaagcaggatcaacaccatgcaatggagtggaaggattcaattgctatttcccactgcagtcatatggattccagccaacaaatggagtgggatatcaaccatatagagtggtggtgctttcattcgaacttcttcatgcaccagcaacagtgtgcggaccaaaaaaatcaacaaatcttgtgaaaaataaatgcgtgaatttc

SEQ ID NO: 6 - DNA, 33 bp, Artificial Sequence
  Synthetic - flag tag
ggcagtggggactataaggacgatgatgacaaa

SEQ ID NO: 7 - DNA, 66 bp, Artificial Sequence
  Synthetic - 2A self-cleaving peptide
ggatctggggctaccaacttcagtctcctcaaacaggccggagacgtggaagaaaatcctgggcct

SEQ ID NO: 8 - DNA, 1698 bp, Artificial Sequence
  Synthetic - influenza hemagglutinin (HA) protein
atgaaagcgaatttgttagttttactgtccgcgttggcggccgcggacgcagacacaatatgtataggctaccatgcgaacaattcaaccgacactgttgacacagtactcgagaagaatgtgacagtgacacactctgttaacctgctcgaagacagccacaacggaaaactatgtagattaaaaggaatagccccactacaattggggaaatgtaacatcgccggatggctcttgggaaacccagaatgcgacccactgcttccagtgagatcatggtcctacattgtagaaacaccaaactctgagaatggaatatgttatccaggagatttcatcgactatgaggagctgagggagcaattgagctcagtgtcatcattcgaaagattcgaaatatttcccaaagaaagctcatggcccaaccacaacacaaacggagtaacggcagcatgctcccatgaggggaaaagcagtttttacagaaatttgctatggctgacggagaaggagggctcatacccaaagctgaaaaattcttatgtgaacaaaaaagggaaagaagtccttgtactgtggggtattcatcacccgcctaacagtaaggaacaacagaatctctatcagaatgaaaatgcttatgtctctgtagtgacttcaaattataacaggagatttaccccggaaatagcagaaagacccaaagtaagagatcaagctgggaggatgaactattactggaccttgctaaaacccggagacacaataatatttgaggcaaatggaaatctaatagcaccaatgtatgctttcgcactgagtagaggctttgggtccggcatcatcacctcaaacgcatcaatgcatgagtgtaacacgaagtgtcaaacacccctgggagctataaacagcagtctcccttaccagaatatacacccagtcacaataggagagtgcccaaaatacgtcaggagtgccaaattgaggatggttacaggactaaggaacactccgtccattcaatccagaggtctatttggagccattgccggttttattgaagggggatggactggaatgatagatggatggtatggttatcatcatcagaatgaacagggatcaggctatgcagcggatcaaaaaagcacacaaaatgccattaacgggattacaaacaaggtgaacactgttatcgagaaaatgaacattcaattcacagctgtgggtaaagaattcaacaaattagaaaaaaggatggaaaatttaaataaaaaagttgatgatggatttctggacatttggacatataatgcagaattgttagttctactggaaaatgaaaggactctggatttccatgactcaaatgtgaagaatctgtatgagaaagtaaaaagccaattaaagaataatgccaaagaaatcggaaatggatgttttgagttctaccacaagtgtgacaatgaatgcatggaaagtgtaagaaatgggacttatgattatcccaaatattcagaagagtcaaagttgaacagggaaaaggtagatggagtgaaattggaatcaatggggatctatcagattctggcgatctactcaactgtcgccagttcactggtgcttttggtctccctgggggcaatcagtttctggatgtgttctaatggatctttgcagtgcagaatatgcatctga

SEQ ID NO: 9 - DNA, 45 bp, Artificial Sequence
  Synthetic - viral 3′ UTR
gattagaatttcagaaatatgaggaaaaacacccttgtttctact

SEQ ID NO: 10 - protein, 40 aa, Artificial Sequence
  Synthetic - portion of a neuraminidase (NA) protein comprising the transmembrane domain and cytoplasmic tail
MNPNQKITTIGSICLVVGLISLILQIGNIISIWISHSIQT

SEQ ID NO: 11 - protein, 223 aa, Artificial Sequence
  Synthetic - receptor binding domain (RBD) of the spike protein from the SARS-CoV-2 Wuhan variant
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 12 - protein, 11 aa, Artificial Sequence
  Synthetic - flag tag
GSGDYKDDDDK

SEQ ID NO: 13 - protein, 22 aa, Artificial Sequence
  Synthetic - 2A self-cleaving peptide
GSGATNFSLLKQAGDVEENPGP

SEQ ID NO: 14 - protein, 565 aa, Artificial Sequence
  Synthetic - influenza hemagglutinin (HA) protein
MKANLLVLLSALAAADADTICIGYHANNSTDTVDTVLEKNVTVTHSVNLLEDSHNGKLCRLKGIAPLQLGKCNIAGWLLGNPECDPLLPVRSWSYIVETPNSENGICYPGDFIDYEELREQLSSVSSFERFEIFPKESSWPNHNTNGVTAACSHEGKSSFYRNLLWLTEKEGSYPKLKNSYVNKKGKEVLVLWGIHHPPNSKEQQNLYQNENAYVSVVTSNYNRRFTPEIAERPKVRDQAGRMNYYWTLLKPGDTIIFEANGNLIAPMYAFALSRGFGSGIITSNASMHECNTKCQTPLGAINSSLPYQNIHPVTIGECPKYVRSAKLRMVTGLRNTPSIQSRGLFGAIAGFIEGGWTGMIDGWYGYHHQNEQGSGYAADQKSTQNAINGITNKVNTVIEKMNIQFTAVGKEFNKLEKRMENLNKKVDDGFLDIWTYNAELLVLLENERTLDFHDSNVKNLYEKVKSQLKNNAKEIGNGCFEFYHKCDNECMESVRNGTYDYPKYSEESKLNREKVDGVKLESMGIYQILAIYSTVASSLVLLVSLGAISFWMCSNGSLQCRICI

SEQ ID NO: 15 - protein, 1273 aa, SARS-CoV-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 16 - DNA, 28 bp, Artificial Sequence
  Synthetic - RT-PCR primer
gtagatgcagcaaaagcaggggaaaata

SEQ ID NO: 17 - DNA, 18 bp, Artificial Sequence
  Synthetic - RT-PCR primer
ccatcctcaatttggcac

SEQ ID NO: 18 - DNA, 20 bp, Artificial Sequence
  Synthetic - RT-PCR primer
agcaaaagcaggggaaaata

SEQ ID NO: 19 - DNA, 20 bp, Artificial Sequence
  Synthetic - RT-PCR primer
gtcttcgagcaggttaacag

SEQ ID NO: 20 - protein, 223 aa, Artificial Sequence
  Synthetic - receptor binding domain (RBD) of the spike protein from the SARS-CoV-2 Beta variant
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNF

SEQ ID NO: 21 - protein, 223 aa, Artificial Sequence
  Synthetic - receptor binding domain (RBD) of the spike protein from the SARS-CoV-2 Gamma variant
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNF

SEQ ID NO: 22 - protein, 223 aa, Artificial Sequence
  Synthetic - receptor binding domain (RBD) of the spike protein from the SARS-CoV-2 Delta variant
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNF

SEQ ID NO: 23 - protein, 223 aa, Artificial Sequence
  Synthetic - receptor binding domain (RBD) of the spike protein from the SARS-CoV-2 Omicron variant
RVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLKSYSFRPTYGVGHQPYRVVVLSFELLHAPATVCGPKKSTNLVKNKCVNF